(19) United States
(12) Patent Application Publication
(10) Pub. No.: US 2026/0283957 A1
(43) Pub. Date: Sep. 24, 2026

(54) Liposome Composition Comprising Farnesol and Method of Use Thereof for Treating Fungal Infection

(71) Applicant: Brown University, Providence, RI (US)

(72) Inventors: Anita Shukla, East Greenwich, RI (US); Camila Carvalho, Providence, RI (US); Veronica LaMastro, Los Angeles, CA (US)

(21) Appl. No.: 19/571,842

(22) Filed: Mar. 19, 2026

Related U.S. Application Data
(60) Provisional application No. 63/774,636, filed on Mar. 19, 2025.

(51) Int. Cl. A61K 9/1273; A61K 9/1277; A61K 9/51; A61K 31/4174; A61K 31/4196; A61K 31/496; A61K 31/506; A61K 49/00; A61P 31/10; B82Y 5/00

(52) U.S. Cl. CPC A61K 9/1273; A61K 9/1277; A61K 9/5123; A61K 9/5138; A61K 9/5192; A61K 31/4174; A61K 31/4196; A61K 31/496; A61K 31/506; A61K 49/0041; A61K 49/0086; A61P 31/10; B82Y 5/00

(57) ABSTRACT

The present disclosure provides a liposome composition comprising a plurality of liposomes, wherein each liposome of the plurality of liposomes comprises a lipid bilayer comprising hydrogenated soy phosphatidylcholine (HSPC), cholesterol (CHOL), and farnesol (FAR). Each liposome may be coated on the outer lipid bilayer with a negatively charged polymer such as poly(acrylic acid) (PAA), heparin, or other polyanions. Each liposome may further encapsulate one or more antifungal therapeutics. Methods of treating or preventing fungal infections, including vulvovaginal candidiasis caused by C. albicans, using the liposome composition are also provided. Methods of making the liposome formulation are further provided.

### CROSS-REFERENCE TO RELATED APPLICATIONS

This application claims priority to U.S. Application No. 63/774,636, filed Mar. 19, 2025, which is hereby incorporated by reference in its entirety.

### STATEMENT OF GOVERNMENT INTERESTS

This invention was made with government support under grant number 1942418, awarded by the National Science Foundation. The government has certain rights in the invention.

### TECHNICAL FIELD

The present disclosure relates to liposome compositions and methods of use thereof for treating fungal infections, and more particularly to liposome compositions comprising hydrogenated soy phosphatidylcholine, cholesterol, and farnesol for inhibiting hyphal formation and methods of use thereof for treating Candida albicans infections including vulvovaginal candidiasis.

### BACKGROUND

Fungal infections represent a substantial public health concern affecting millions of individuals worldwide (Rayens E. and Norris K. A. Open Forum Infect Dis. (2022)). Among these infections, vulvovaginal candidiasis (VVC) is a common fungal infection among reproductive age women, with approximately 75% of women being diagnosed with VVC at least once during their lifetime (Neal C. M. and Martens M. G. SAGE Open Medicine (2022)). The condition is caused by an overgrowth of Candida albicans (C. albicans), an opportunistic fungal pathogen that normally exists as a commensal organism in the human body. Factors such as immune dysfunction and misuse or overuse of antibiotics can contribute to the development of these infections. Recurrent VVC affects a substantial portion of women, with projections indicating that 158 million women worldwide may experience recurrent VVC by 2030 (Denning D. W. et al. Lancet Infect Dis (2018)). Women with recurrent VVC have a higher risk of developing systemic infections, making effective treatment approaches desirable.

C. albicans exhibits morphological plasticity, existing in yeast, pseudohyphal, and hyphal forms. The transition from the commensal yeast form to the pathogenic hyphal form is associated with increased virulence. This yeast-to-hyphal transition enables the fungi to physically penetrate mucosal cells by forming invasion pockets and allows the organism to breach macrophage membranes, causing lysis. The filamentous structure of hyphal cells enables evasion of the host immune system. Importantly, the pathogenic hyphal form represents the predominant morphology within C. albicans biofilms, which are hydrated three-dimensional communities of fungal cells surrounded by secreted extracellular polymeric substances. These biofilm structures complicate the treatment of fungal diseases by reducing susceptibility to antifungal drugs due to the presence of the extracellular polymeric substances, increased expression of drug efflux pumps, and altered drug targets.

Hyphal cells of C. albicans secrete candidalysin, a cytolytic peptide toxin that contributes to tissue damage during infection. The damaging effects of candidalysin upon its release from hyphal cells represent an additional challenge in managing C. albicans infections.

Current treatment approaches for VVC include oral doses of fluconazole and topical creams or ointments containing antifungal agents. However, these conventional treatments may not adequately address the underlying morphological transitions that contribute to pathogenicity. Antifungal drugs commonly used include azoles such as fluconazole, posaconazole, and voriconazole, as well as polyenes and echinocandins. Many of these antifungal drugs are hydrophobic, presenting challenges for formulation and delivery.

Biomaterials have been used for treatment of fungal infections to improve the delivery of antifungal drugs and reduce drug-related toxicity. Nanoparticles have emerged as a therapeutic avenue for the treatment of fungal infections because of their tunable properties and potential for localized and targeted therapeutic delivery. Liposomes, which are spherical lipid vesicles comprised of amphiphilic phospholipids that assemble into a phospholipid bilayer surrounding an aqueous core, can encapsulate both hydrophobic and hydrophilic therapeutics due to their structure. As many antifungal drugs are hydrophobic, liposomes allow for enhanced solubility of these treatments.

Farnesol is a quorum-sensing molecule secreted by C. albicans to regulate hyphal growth (Batliner M. et al. Host-Microbial Interactions (2024), Hornby J. M. et al. Applied and Environmental Microbiology (2001), Ramage G. et al. Applied and Environmental Microbiology (2002)). This compound has been identified as one of the lipids found in extracellular vesicles secreted by C. albicans. Farnesol has demonstrated antihyphal properties against C. albicans (Honorato L. et al. Mycology (2022)), with the ability to reduce the yeast-to-hyphal transition and biofilm formation (Rodrigues C. F. & Černiková L. Genes (2020)).

While there has been interest in using liposomes for treatment of fungal infections, investigations into the impact of liposome composition on liposome-hyphae interactions remain limited. Some studies have shown that liposome formulation affects interaction with C. albicans biofilms, with saturated, positively charged liposomes demonstrating enhanced interaction (Smistad G. et al., Journal of Liposome Research (2011)). Some liposomes have also been shown to preferentially interact with hyphal cells (LaMastro V. et al. J. Biomed. Mater. Res. (2023), Chavan N. L. et al. Mol. Pharm. (2012)). However, nanoparticle formulations designed to prevent the formation of hyphal cells during infection and to neutralize the damaging effects of candidalysin, as a therapeutic avenue, represent an area for further development.

Accordingly, there remains a need for improved compositions and methods for treating fungal infections, particularly those that can address hyphal formation and the associated pathogenic mechanisms of C. albicans.

### SUMMARY

According to one aspect of the present disclosure, a liposome composition is provided. The liposome composition comprises a plurality of liposomes wherein each liposome of the plurality of liposomes comprises a lipid bilayer comprising hydrogenated soy phosphatidylcholine (HSPC), cholesterol (CHOL), and/or farnesol (FAR).

According to other aspects of the present disclosure, the liposome composition may include one or more of the following features. In certain embodiments, the molar ratios of HSPC:CHOL:FAR include HSPC: about 78-85 mol %, CHOL: about 15-40 mol %, and FAR: about 0-5 mol %. In one embodiment, the molar ratio of HSPC:CHOL:FAR is about 85:15:0. In another embodiment, the molar ratio of HSPC:CHOL:FAR is about 83:15:2. In yet another embodiment, the molar ratio of HSPC:CHOL:FAR is about 80:15:5. In still another embodiment, the molar ratio of HSPC:CHOL:FAR is about 78:15:7. Each liposome of the plurality of liposomes may be further coated on the outer lipid bilayer with a poly(acrylic acid) (PAA), heparin, or other polyanions. Each liposome of the plurality of liposomes may have a size of about 50 nm to about 200 nm in diameter. Each liposome of the plurality of liposomes may have a size of about 100 nm to about 150 nm in diameter. Each liposome of the plurality of liposomes may be further encapsulated with one or more antifungal therapeutics including azole, polyene, and/or echinocandin. The antifungal therapeutics may be encapsulated within the lipid bilayer and/or the hydrophilic core of each liposome of the plurality of liposomes. In some embodiments, the azole may comprise posaconazole, fluconazole, and voriconazole. In one embodiment, each liposome of the plurality of liposomes may be further labeled with a fluorescent dye.

According to another aspect of the present disclosure, a pharmaceutical composition is provided. The pharmaceutical composition comprises a liposome composition comprising a plurality of liposomes wherein each liposome of the plurality of liposomes comprises a lipid bilayer comprising hydrogenated soy phosphatidylcholine (HSPC), cholesterol (CHOL), and/or farnesol (FAR).

According to other aspects of the present disclosure, the pharmaceutical composition may further comprise a pharmaceutically acceptable excipient.

According to another aspect of the present disclosure, a kit is provided. The kit comprises a liposome composition comprising a plurality of liposomes wherein each liposome of the plurality of liposomes comprises a lipid bilayer comprising hydrogenated soy phosphatidylcholine (HSPC), cholesterol (CHOL), and/or farnesol (FAR). The kit further comprises technical instructions providing information on administration and dosage of the composition.

According to another aspect of the present disclosure, a method of treating or preventing a fungal infection within a subject in need thereof is provided. In one embodiment, the method comprises administering to the subject a therapeutically effective amount of a liposome composition. Each liposome of the liposome composition comprises a lipid bilayer comprising hydrogenated soy phosphatidylcholine (HSPC), cholesterol (CHOL), and/or farnesol (FAR).

According to other aspects of the present disclosure, the method may include one or more of the following features. In certain embodiments, the molar ratios of HSPC:CHOL:FAR include HSPC: about 78-85 mol %, CHOL: about 15-40 mol %, and FAR: about 0-5 mol %. In one embodiment, the molar ratio of HSPC:CHOL:FAR is about 85:15:0. In another embodiment, the molar ratio of HSPC:CHOL:FAR is about 83:15:2. In yet another embodiment, the molar ratio of HSPC:CHOL:FAR is about 80:15:5. Each liposome of the plurality of liposomes may be further coated on the outer lipid bilayer with a poly(acrylic acid) (PAA), heparin, or other polyanions. Each liposome of the plurality of liposomes may have a size of about 50 nm to about 200 nm in diameter. Each liposome of the plurality of liposomes may have a size of about 100 nm to about 150 nm in diameter. Each liposome of the plurality of liposomes may be further encapsulated with one or more antifungal therapeutics including azole, polyene, and/or echinocandin. The antifungal therapeutics may be encapsulated within the lipid bilayer and/or the hydrophilic core of each liposome of the plurality of liposomes. In some embodiments, the azole may comprise posaconazole, fluconazole, and voriconazole. In one embodiment, each liposome of the plurality of liposomes may be further labeled with a fluorescent dye.

The liposome composition may be administered to the subject by a route selected from the group consisting of mucosal, intranasal, intravaginal, intramuscular, intravenous, subcutaneous, intradermal, subdermal, and transdermal routes of administration. The subject may be a human. The fungal infection may be yeast infection. The yeast infection may be vulvovaginal candidiasis (VVC), caused by an overgrowth of C. albicans. The liposome composition may inhibit hyphal formation or yeast-to-hyphal transition in C. albicans. The FAR may interact with and neutralize candidalysin peptide secreted by hyphae, thereby preventing or inhibiting hyphal formation or yeast-to-hyphal transition in C. albicans. The FAR may interact with and neutralize candidalysin peptide secreted by hyphae, thereby neutralizing the damaging effects of candidalysin peptide upon its release from hyphal cells. The liposome composition may enhance fungal clearance in vitro and in vivo. The fungal infection may be prevented, reduced, inhibited or eradicated. After administration, the liposome composition may contact fungal biofilm in the subject and disrupt the fungal biofilm. The PAA may neutralize and degrade the candidalysin peptide secreted by hyphae.

According to another aspect of the present disclosure, a method of making a liposome formulation is provided. The method comprises providing a lipid film over a drug film, wherein the lipid film comprises a desired molar ratio of HSPC, CHOL, and FAR, and wherein the drug film comprises antifungal therapeutics. The method further comprises hydrating the lipid films with 10 mM HEPES buffer (pH 7.4) to form a lipid suspension with a total lipid concentration of 2.5 mg/mL, followed by vigorous vortexing to generate multilamellar vesicles. The method further comprises sonicating the lipid suspension for 15 min at 65° C. The method further comprises extruding multiple times, e.g., 5-20 times, through a 100 nm polycarbonate membrane to obtain uniformly sized liposomes.

According to other aspects of the present disclosure, the method of making a liposome formulation may include one or more of the following features. The liposome formulation may be further labeled with a fluorescent dye by incorporating 0.1% (w/w) 1,2-distearoyl-sn-glycero-3-phosphoethanolamine-N-(Lissamine rhodamine B sulfonyl) (LRB). The liposome formulation may be further treated with 5% (w/w) 1,2-dioleoyl-3-trimethylammonium-propane (DOTAP) to generate a net positive surface charge. DOTAP-containing liposomes (3 mM) may be added dropwise to PAA suspension (0.08% w/v), at a 1:4 v/v ratio, at a speed of 0.1 mL/min under constant stirring at 700 rpm with a micro stir bar.

Fluorescent dyes for labeling the liposomes are known to one skilled in the art. Suitable fluorescent dyes for incorporation into the liposome according to certain embodiments of the present disclosure include, but are not limited to, lipophilic membrane dyes such as 1,1′-dioctadecyl-3,3,3′,3′-tetramethylindocarbocyanine perchlorate (DiI), 3,3′-dioctadecyloxacarbocyanine perchlorate (DiO), and 1,1′-dioctadecyl-3,3,3′,3′-tetramethylindodicarbocyanine perchlorate (DiD), and conjugatable dyes such as rhodamine B and cyanine5 (Cy5).

According to another aspect of the present disclosure, a method of treating a fungal infection in a subject in need thereof is provided. The method comprises administrating to the subject a pharmaceutical composition comprising a liposome composition comprising a plurality of liposomes wherein each liposome of the plurality of liposomes comprises a lipid bilayer comprising hydrogenated soy phosphatidylcholine (HSPC), cholesterol (CHOL), and/or farnesol (FAR). The fungal infection is prevented, reduced, inhibited or eradicated.

According to another aspect of the present disclosure, a method of treating a fungal biofilm within a subject in need thereof is provided. The method comprises administrating to the subject a pharmaceutical composition comprising a liposome composition comprising a plurality of liposomes wherein each liposome of the plurality of liposomes comprises a lipid bilayer comprising hydrogenated soy phosphatidylcholine (HSPC), cholesterol (CHOL), and/or farnesol (FAR). The farnesol, antifungal therapeutics, and PAA in the composition prevent hyphal formation, interact with candidalysin peptide secreted by the fungal hyphae, and prevent or disrupt the formation of the fungal biofilm.

The foregoing general description of the illustrative embodiments and the following detailed description thereof are merely exemplary aspects of the teachings of this disclosure and are not restrictive.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawing in which:

FIG. 1 illustrates a schematic representation of liposome compositions and liposome-based strategies to inhibit hyphal formation and neutralize candidalysin. The top panel depicts antihyphal liposomes composed of HSPC and cholesterol and incorporating farnesol inhibit the yeast-to-hypha transition of C. albicans, thereby preventing filamentation. The bottom panel depicts candidalysin-neutralizing liposomes, formulated with cationic lipids (e.g., DOTAP) and a polymer coating, interact with hyphae and neutralize secreted candidalysin at the epithelial interface, reducing toxin-mediated cell damage.

FIG. 2 illustrates a schematic representation of dual therapeutic mechanisms: inhibition of hyphal morphogenesis and neutralization of hypha-associated candidalysin within the invasion pocket, ultimately limiting epithelial damage.

FIG. 3 illustrates an overview of the morphological transition schematic of C. albicans from yeast to pseudohyphae and hyphae and its contribution to virulence. The yeast-to-hypha transition promotes (top) epithelial cell damage through invasive growth and invasion pocket formation, (middle) immune cell evasion via filamentation-associated escape from phagocytes, and (bottom) biofilm formation supported by secretion of extracellular polymeric substances (EPS). Targeting hyphal formation may therefore represent a therapeutic strategy to reduce C. albicans pathogenicity.

FIGS. 4A-4C depict bar graph characterization results showing the stability of blank and drug-loaded antihyphal liposomes over 28 days during storage at 4° C. Liposomes composed of different lipid ratios (85:15 HSPC:CHOL, 83:15:2 HSPC:CHOL:FAR, and 80:15:5 HSPC:CHOL:FAR) were evaluated either as blank vesicles or loaded with fluconazole (FLU) or miconazole (MCZ). FIG. 4A shows the hydrodynamic diameter, which remained relatively stable (~110-150 nm) across all formulations throughout the study, indicating minimal changes in particle size during storage. FIG. 4B presents the polydispersity index (PDI). Most formulations maintained PDI values below ~0.20, indicating relatively homogeneous particle populations, although some statistically significant variations were observed at specific time points. FIG. 4C shows the ζ-potential, which ranged from approximately −5 to +12 mV depending on formulation and drug loading. Minor fluctuations were observed over time, but overall values remained consistent with stable colloidal dispersions. Measurements were taken weekly and values are expressed as mean±SD (n=3). Statistical analysis was performed using a two-way ANOVA with Tukey's post hoc analysis; n=4; α=0.05; and *p<0.05, **p<0.01, ***p<0.001, **** p<0.0001.

FIGS. 5A-5B depict experimental quantitative analysis of farnesol (FAR) encapsulation via HPLC. FIG. 5A shows representative chromatograms of free farnesol (FAR), 2% FAR-loaded liposomes (FAR-LIPO), 5% FAR-LIPO, and blank liposome controls. All FAR-containing formulations exhibit a characteristic absorbance peak at a retention time of approximately 6.2 min. FIG. 5B depicts a calibration curve showing relationship between known FAR concentrations and the integrated Area Under the Curve (AUC).

FIGS. 6A-6B depict hyphal inhibition assay results of confocal imaging of antihyphal liposomes incubated with C. albicans SC5314. FIG. 6A depicts representative images showing C. albicans distinct morphologies (yeast, pseudohyphae, and hyphae) upon interaction with antihyphal liposome formulations. Control formulations (60:40 HSPC:CHOL, non-antihyphal) and untreated controls without liposome exposure are also shown, including samples incubated at 30° C. (yeast-only growth) and 37° C. (hyphae-inducing conditions). FIG. 6B depicts quantification of yeast vs. hyphal cell counts upon liposome treatment. Statistical analysis was performed using a two-way ANOVA with Tukey's post hoc analysis; n=4; α=0.05; and **p<0.01, ***p<0.001, ****p<0.0001.

FIGS. 7A-7B depict bar graph results showing antifungal activity of drug-loaded liposomes. Normalized planktonic growth of C. albicans SC5314 following exposure to free drug, blank liposomes, and antihyphal liposomes loaded with fluconazole (FLU) (FIG. 7A) or miconazole (MCZ) (FIG. 7B).

FIGS. 8A-8F depict bar graph results showing antifungal activity of free drugs and drug-loaded liposomes against planktonic C. albicans SC5314. Normalized growth was measured after exposure to free posaconazole, fluconazole, and voriconazole (FIGS. 8A-8C), as well as blank liposomes and antihyphal liposomes loaded with posaconazole, fluconazole, and voriconazole (FIGS. 8D-8F), respectively.

FIG. 9 depicts bar graph results showing inhibition of biofilm biomass assessed by crystal violet staining upon exposure to blank and drug-loaded antihyphal liposomes.

FIG. 10 depicts bar graph results showing cytocompatibility of antihyphal liposomes using a Cell Counting Kit-8 (CCK-8) assay, illustrating vaginal epithelial cell viability after treatment with blank and drug-loaded liposomes.

FIGS. 11A-11D depict a schematic representation of the well plate assay and corresponding results evaluating cytotoxicity in three mammalian cell types following exposure to candidalysin and different polyanions, assessed using a lactate dehydrogenase (LDH) release assay to determine whether the polyanions neutralize candidalysin-induced toxicity. FIG. 11A depicts a schematic summary of the experimental design. Bar graph results of cytotoxicity to fibroblasts (FIG. 11B), HUVECs (FIG. 11C), and vaginal epithelial cells (FIG. 11D) upon simultaneous exposure to candidalysin (250 g/mL) and polymer of interest, as measured through release of intracellular lactate dehydrogenase (LDH). Dotted line represents normal LDH release of untreated controls. Statistical analysis was performed using a two-way ANOVA with Tukey's post hoc analysis; n=4; α=0.05; and *p<0.05, **p<0.01, ***p<0.001, ****p<0.0001.

FIGS. 12A-12C depict bar graph results of polymer-coated liposome characterization showing hydrodynamic diameter, ζ-potential, and polydispersity index for uncoated and PAA-coated liposomes. Data presented as mean±SD (n=3 biological replicates). Statistical significance was determined by two-way ANOVA followed by Tukey's multiple comparisons test (α=0.05). Asterisks denote significant differences (****p<0.0001). FIG. 12A shows the hydrodynamic diameter (nm) of the liposomes. PAA-coated liposomes exhibited a significantly larger hydrodynamic diameter compared to bare liposomes, consistent with the presence of a polymer layer on the liposome surface. FIG. 12B presents the PDI. Although PAA-coated liposomes displayed a slightly higher PDI than bare liposomes, the difference was not statistically significant, indicating that polymer coating did not substantially affect particle size distribution. FIG. 12C shows the ζ-potential (mV) of the formulations. A significant shift from a positive ζ-potential for bare liposomes to a negative ζ-potential after PAA coating was observed, confirming successful adsorption of the anionic polymer onto the liposome surface.

FIG. 13 depicts bar graph results of comparative efficacy data of liposome formulations, showing PAA coating stability under varying pH conditions. Data are presented as mean±SD (n=3). Statistical significance was determined by two-way ANOVA followed by Tukey's multiple comparisons test (α=0.05). Asterisks denote significant differences (**** p<0.0001). The PDI for each formulation is indicated above the corresponding bar.

### DETAILED DESCRIPTION

### Definitions

For convenience, the meaning of some terms and phrases used in the specification, examples, and appended claims is provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an,” and “the” include plural referents unless the context dictates otherwise.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The abbreviation “e.g.” is derived from the Latin exempli gratia and is used herein to indicate a non-limiting example. Thus, the abbreviation “e.g.” is synonymous with the term “for example.”

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, a description referring to “about X” includes a description of “X”.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein, the term “consisting essentially of” refers to those elements required for a given embodiment. The term permits the presence of additional elements that do not materially affect the basic and novel or functional characteristic(s) of that embodiment of the invention.

As used herein, the phrase “therapeutically effective amount”, “effective amount” or “effective dose” refers to an amount that provides a therapeutic or aesthetic benefit in the treatment, prevention, or management of a fungal infection, e.g., an amount that provides a statistically significant decrease in at least one symptom, sign, or marker of a fungal infection. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a fungal infection, delay or slowing of fungal infection, and an increased lifespan as compared to that expected in the absence of treatment.

As used herein, the term “administering” refers to the placement of a liposome formulation, as disclosed herein, into a subject by a method or route that results in at least partial delivery of the agent at a desired site. Pharmaceutical compositions comprising the liposome compositions disclosed herein can be administered by any appropriate route, which results in an effective treatment in the subject.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravaginal, intravascular, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such a level.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein, and as such can vary. The terminology used herein is to describe particular embodiments only and is not intended to limit the scope of the present invention, which is defined solely by the claims. Definitions of common terms in immunology and molecular biology can be found in The Merck Manual of Diagnosis and Therapy, 19th (2011); The Encyclopedia of Molecular Cell Biology and Molecular Medicine, (1999-2012); Molecular Biology and Biotechnology: A Comprehensive Desk Reference, (1995); Immunology (2006); Janeway's Immunobiology (2014); Lewin's Genes XI (2014); Molecular CLONING: A LABORATORY MANUAL, 4th ed. (2012); Basic Methods in Molecular Biology (2012); Laboratory Methods in Enzymology: DNA (2013); Current Protocols in Molecular Biology (CPMB) (2014); Current Protocols in Protein Science (CPPS) (2005); and Current Protocols in Immunology (CPI) (2003), the contents of which are all incorporated by reference herein in their entireties.

In some embodiments, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and animals resulting from such processes.

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to liposome compositions and formulations, pharmaceutical compositions, and methods for treating fungal infections, including infections caused by C. albicans. C. albicans is a fungal organism that can transition from a commensal state to a pathogenic state, with the yeast-to-hyphae transition representing a characteristic feature of pathogenicity and virulence. Hyphal cells enable fungal penetration of mucosal tissues, evasion of host immune responses, and formation of biofilms that reduce susceptibility to antifungal treatments.

Liposome compositions described herein may comprise a lipid bilayer containing hydrogenated soy phosphatidylcholine (HSPC), cholesterol (CHOL), and/or farnesol (FAR). Farnesol is a quorum-sensing molecule that C. albicans secretes to regulate hyphal growth. Incorporation of farnesol into liposome formulations may inhibit the yeast-to-hyphal transition in C. albicans, thereby reducing pathogenicity, virulence, and improving treatment outcomes. The liposome compositions may encapsulate one or more antifungal therapeutics within the lipid bilayer to provide enhanced delivery of hydrophobic drugs. The liposome compositions may further encapsulate one or more antifungal therapeutics within the hydrophilic core in combination with encapsulating one or more antifungal therapeutics within the lipid bilayer.

Antifungal therapeutics, drugs and/or agents are known to one skilled in the art. Additional antifungal agents suitable for incorporation into the liposome according to some embodiments of the present disclosure, including both hydrophobic and hydrophilic compounds, include, but are not limited to, hydrophobic drugs such as amphotericin B, itraconazole, miconazole, posaconazole, voriconazole, and clotrimazole; as well as relatively more hydrophilic agents such as fluconazole, ketoconazole, caspofungin, micafungin, and anidulafungin.

The present disclosure provides a pharmaceutical composition comprising the liposomes as described herein. The pharmaceutical composition may further comprise a pharmaceutically acceptable excipient.

Pharmaceutically acceptable excipients for the liposomes are known in the art. They function to form stable bilayer structures, protect the encapsulated drug, and manage the physicochemical properties (e.g., fluidity and/or surface charge) of the vesicular system. The most common excipients include phospholipids, cholesterol, sterols, pegylated lipids, surfactants, and various stabilization agents. Examples of structural lipid excipients include phospholipids which are the primary bilayer-forming components; natural phospholipids such as egg phosphatidylcholine (EPC) and soybean phosphatidylcholine (SPC). Examples of synthetic/hydrogenated phospholipids include hydrogenated soybean phosphatidylcholine (HSPC), dipalmitoylphosphatidylcholine (DPPC), distearoylphosphatidylcholine (DSPC), and dimyristoylphosphatidylcholine (DMPC). Examples of sphingolipids include sphingomyelin (SM) used to enhance stability, particularly in acidic environments, and to reduce water permeability. Further, cholesterol is a crucial component (often 30-50 mol %) that fits between phospholipids, improving bilayer rigidity, reducing permeability (leaky-ness), and increasing stability. Negatively charged lipids are used to enhance stability via electrostatic repulsion, reducing liposome aggregation. Examples include dipalmitoylphosphatidylglycerol (DPPG), dimyristoylphosphatidylglycerol (DMPG), and phosphatidylserine (PS). Examples of stabilizing and “Stealth” excipients include PEGylated Lipids (Stealth Agents). For example, polyethylene glycol (PEG) derivatives, such as (carbonyl-methoxypolyethylene glycol 2000)-1,2-distearoyl-sn-glycero-3-phosphoethanolamine (MPEG-DSPE), are conjugated to the surface to prevent detection by the immune system and extending circulation time. Additional antioxidants can be used to reduce the oxidation of lipids and cholesterol, including tocopherol (Vitamin E), butylated hydroxytoluene (BHT), and ascorbyl palmitate. Cryoprotectants can be used during lyophilization (freeze-drying) to protect liposome integrity, including sucrose, trehalose, and glycerol.

The present disclosure provides surface-modifying and coating agents for the liposomes. In some embodiments, polymers including chitosan, pectin, alginate, and gelatin are used to coat liposomes. Surfactants are also used including non-ionic surfactants such as polysorbate 80 (Tween 80) and bile salts (e.g., sodium cholate) can be used to form deformable liposomes or “bilosomes” for enhanced penetration.

The present disclosure provides commonly used buffers and solvents for use with the liposomes and liposome formulations. These buffers and solvents are known in the art and can be adjusted according to a specific situation. For example, buffers include phosphate buffered saline (PBS) or citrate buffers and are used to maintain pH (often around 6.5-7.4) and ionic strength, essential for maintaining bilayer structure of the liposomes. In addition, solvents such as ethanol is commonly used in manufacturing for lipid dissolution and is generally considered safe and acceptable.

The excipients provided by the present disclosure must comply with current pharmacopoeias (USP-NF, Ph.Eur., JP) and be of pharmaceutical grade to ensure safety and stability. (Upendra Bulbake, Sindhu Doppalapudi, Nagavendra Kommineni, Wahid Khan; “Liposomal Formulations in Clinical Use: An Updated Review”, Pharmaceutics, 2017, Mar. 27; 9(2):12; Changguang Wang, Prabhath L Gamage, Wenlei Jiang, Thilak Mudalige; “Excipient-related impurities in liposome drug products”, International Journal of Pharmaceutics, Volume 657, May 25, 2024).

In some embodiments, the liposome compositions may be coated with a polymer on the outer lipid bilayer surface. Candidalysin is a cytolytic peptide toxin secreted by C. albicans hyphae that causes epithelial damage and activates host immune responses. Polymer coatings may interact with and neutralize candidalysin peptide, thereby preventing or reducing the damaging effects of candidalysin upon release from hyphal cells. Negatively charged polymers, such as poly(acrylic acid) (PAA), heparin, or other polyanions, may electrostatically interact with candidalysin, which carries a positive charge at physiological pH.

Additional polyanions according to certain embodiments of the present disclosure include, but are not limited to, chondroitin sulfate, dextran sulfate, alginate, and hyaluronic acid.

The liposome compositions described herein may provide a dual-action approach for treating fungal infections. The farnesol component of the lipid bilayer may contribute to inhibition of hyphal formation; however, farnesol alone does not appear to be the sole driver of hyphal inhibition, as stiff formulations such as 85:15 HSPC:CHOL exhibit antihyphal activity comparable to farnesol-containing formulations. The polymer coating may neutralize candidalysin released by pre-existing hyphae. This combination may enhance fungal clearance and reduce the need for high antifungal doses. The liposome compositions may be formulated as pharmaceutical compositions for administration to subjects suffering from fungal infections, including vulvovaginal candidiasis caused by overgrowth of C. albicans.

According to one aspect, the present disclosure provides a treatment methodology for administering the liposome composition to a subject in need thereof. The liposome composition may be administered to the subject by a route selected from the group consisting of mucosal, intranasal, intravaginal, intramuscular, intravenous, subcutaneous, intradermal, subdermal, and transdermal routes of administration. The selection of administration route may depend on the location and type of fungal infection being treated.

In one embodiment, the liposome composition may be administered for treatment of vulvovaginal candidiasis (VVC). Vulvovaginal candidiasis is a common fungal infection among reproductive age women caused by an overgrowth of C. albicans. Approximately 75% of women are diagnosed with VVC at least once during their lifetime. In some cases, the yeast infection is vulvovaginal candidiasis (VVC). The intravaginal route of administration may be selected for treatment of VVC to deliver the liposome composition directly to the site of infection.

In other embodiments, the mucosal route of administration may be selected for treatment of fungal infections affecting mucosal surfaces. The intranasal route of administration may be selected for treatment of fungal infections affecting the nasal passages or sinuses. The intramuscular, intravenous, subcutaneous, intradermal, subdermal, and transdermal routes of administration may be selected for systemic delivery of the liposome composition or for treatment of fungal infections at various anatomical sites. The liposome composition may provide localized delivery of farnesol and encapsulated antifungal therapeutics to inhibit hyphal formation and treat the fungal infection.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples are set forth as being representative of the present disclosure. These examples are not to be construed as limiting the scope of the present disclosure, as these and other equivalent embodiments will be apparent in view of the present disclosure, figures, and accompanying claims.

### EXAMPLES

### Example I

Investigating the Nanoparticle-Mediated Inhibition of the Yeast-to-Hyphal Transition in C. albicans

In one embodiment, the present disclosure investigates the nanoparticle liposome mediated inhibition of the yeast-to-hyphal transition in C. albicans. The liposome composition includes a plurality of liposomes. Each liposome of the plurality of liposomes comprises a lipid bilayer comprising hydrogenated soy phosphatidylcholine (HSPC), cholesterol (CHOL), and farnesol (FAR). The liposome composition may also be configured to provide dual therapeutic mechanisms for treating fungal infections, including inhibition of hyphal formation and neutralization of candidalysin.

In one embodiment, the phospholipids being used has the following structure: Hydro Soy PC (HSPC)

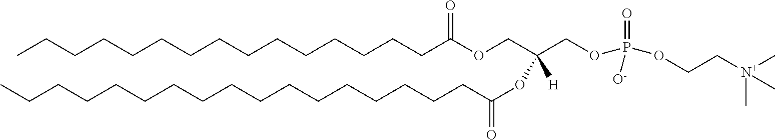 [SMILES: CCCCCCCCCCCCCCCCCC(=O)O[C@H](COC(=O)CCCCCCCCCCCCCCC)COP(=O)([O-])OCC[N+](C)(C)C]

In one embodiment, the farnesol being used has the following structure: Farnesol (FAR)

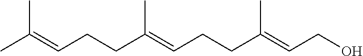 [SMILES: CC(C)=CCC/C(C)=C/CC/C(C)=C/CO]

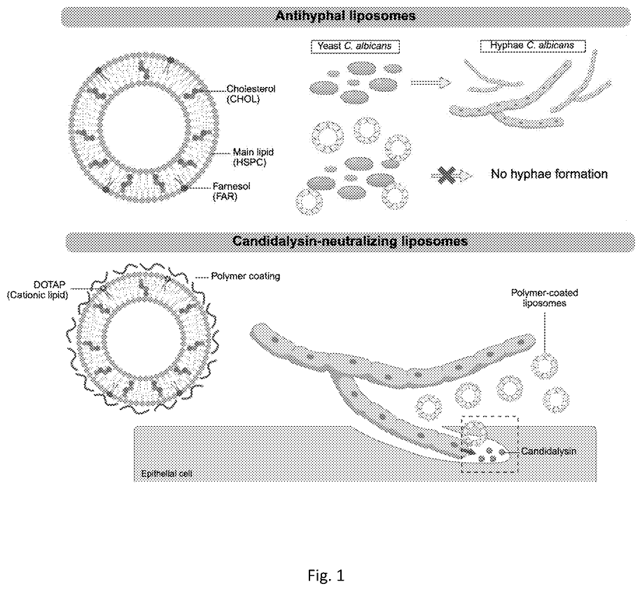

As shown in FIG. 1, antihyphal liposomes composed of HSPC, cholesterol, and farnesol inhibited the yeast-to-hypha transition of C. albicans, thereby preventing filamentation. The liposome formulation included HSPC with saturated acyl chains, which have been shown to inhibit C. albicans hyphal formation. Cholesterol was incorporated into the lipid bilayer to modulate membrane fluidity and mechanical rigidity, as well as improve liposome stability and retention of hydrophobic cargo such as azole drugs.

In another embodiment, the present disclosure provides candidalysin-neutralizing liposomes, which was formulated with cationic lipids and a polymer coating. The candidalysin-neutralizing liposomes interacted with hyphae and neutralized secreted candidalysin at an epithelial interface, reducing toxin-mediated cell damage. Candidalysin is a secreted, pore-forming peptide toxin produced by C. albicans during hyphal growth and is a mediator of epithelial damage and host immune activation.

The liposome composition thus provided a dual-action nanoparticle system. In some embodiments, the antihyphal liposomes prevented hyphal formation through the action of HSPC and farnesol incorporated within the lipid bilayer. In other embodiments, the candidalysin-neutralizing liposomes neutralized candidalysin released by pre-existing hyphae through electrostatic interactions between a polymer coating and the positively charged candidalysin peptide.

In one embodiment, the present disclosure provides a method of making a liposome formulation. The method includes providing a lipid film comprising a desired molar ratio of hydrogenated soy phosphatidylcholine (HSPC), cholesterol (CHOL), and/or farnesol (FAR). The liposomes were prepared using a thin-film hydration method followed by extrusion. The extrusion was conducted multiple times, such as 5, 6, 7, 8, 9, 10, 11, 12, 13, 14, 15, 16, 17, 18, 19, 20 or more times. The lipid film was prepared from lipid stock solutions dissolved in chloroform. The organic solvent was removed under reduced pressure using a rotary evaporator to form a dried lipid film.

In one embodiment, the dried lipid films were placed under vacuum in a desiccator overnight to ensure complete removal of residual solvent before hydration. The method further included hydrating the lipid film with a buffer to form a lipid suspension. In some embodiments, the lipid films were hydrated with 10 mM HEPES buffer at pH 7.4 to achieve a final total lipid concentration of 2.5 mg/mL. Following hydration, vigorous vortexing was performed to generate multilamellar vesicles.

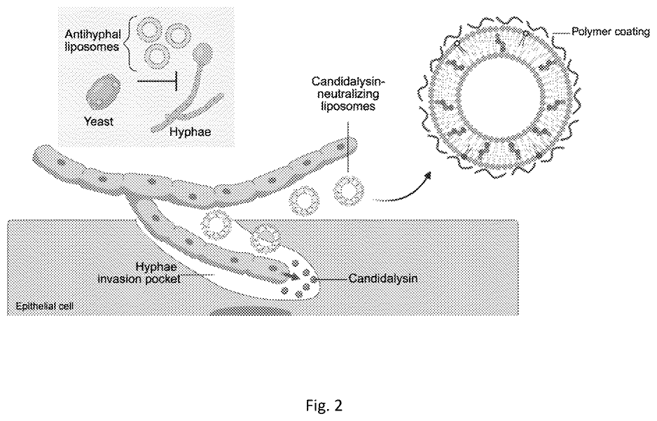

As shown in FIG. 2, the method included sonicating the lipid suspension. In some embodiments, the liposome suspensions were sonicated for 15 minutes at 65° C. prior to extrusion. Extrusion was conducted by extruding the lipid suspension multiple times through a polycarbonate membrane to obtain uniformly sized liposomes. In some embodiments, the liposomes were extruded through a 100 nm polycarbonate membrane to obtain uniformly sized liposomes with a target hydrodynamic diameter of approximately 100 nm. The liposomes may be extruded through the polycarbonate membrane multiple times, such as 5, 6, 7, 8, 9, 10, 11, 12, 13, 14, 15, 16, 17, 18, 19, 20 or more times to achieve uniform sizing.

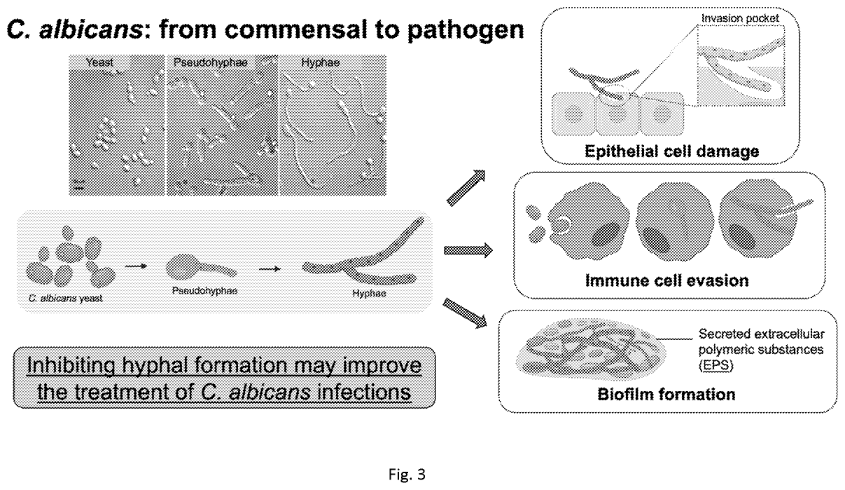

In one embodiment, the present disclosure provides a morphological transition schematic of C. albicans from yeast to pseudohyphae and hyphae and the contribution of the morphological transition to virulence. The morphological transition schematic depicts how C. albicans exists in multiple morphological forms, including yeast cells, pseudohyphae, and true hyphae. The transition from a commensal to a pathogenic state may be characterized by the yeast-to-hyphal transition, which enables the fungi to physically penetrate mucosal cells. FIG. 3.

In another embodiment, the present disclosure provides a schematic showing that the yeast-to-hyphal transition promotes epithelial cell damage through invasive growth and invasion pocket formation. The morphological transition schematic shows that hyphal cells form an invasion pocket at the epithelial interface, allowing C. albicans to breach epithelial barriers. The filamentous structure of hyphal cells enables evasion of the host immune system. As further shown in FIG. 3, C. albicans hyphal growth allows the fungi to breach macrophage membranes, causing lysis and enabling escape from phagocytes.

The morphological transition schematic further illustrates that the pathogenic hyphal form is one of the main components of biofilms. Biofilms are highly hydrated three-dimensional communities of fungal cells surrounded by secreted extracellular polymeric substances (EPS). The biofilm structures may complicate treatment of fungal diseases by reducing susceptibility to antifungal drugs due to the presence of EPS, increased expression of drug efflux pumps, and altered drug targets. FIG. 3

In one embodiment, the present disclosure provides a method of treating or preventing a fungal infection within a subject in need thereof comprises administering to the subject a therapeutically effective amount of a liposome composition. As described previously, each liposome of the liposome composition comprises a lipid bilayer comprising hydrogenated soy phosphatidylcholine (HSPC), cholesterol (CHOL), and/or farnesol (FAR). In some embodiments, the fungal infection is a yeast infection caused by C. albicans.

In another embodiment, the present disclosure provides that the liposome composition inhibits hyphal formation or yeast-to-hyphal transition in C. albicans. Farnesol (FAR) is a quorum-sensing molecule secreted by C. albicans to regulate hyphal growth. Farnesol is one of the primary lipids found in extracellular vesicles secreted by C. albicans. By incorporating farnesol into the lipid bilayer of the liposomes, the liposome composition delivers farnesol to C. albicans cells to suppress the yeast-to-hyphal transition. However, farnesol was not strictly required for antihyphal activity, as stiff liposome formulations such as 85:15 HSPC:CHOL also exhibited significant inhibition of hyphal formation. Inhibiting hyphal formation may reduce the pathogenicity of C. albicans by preventing epithelial cell damage, immune cell evasion, and biofilm formation as depicted in the morphological transition schematic FIG. 3.

The method for FIG. 3 of the present disclosure provides that the liposomes were fabricated using a thin-film hydration method followed by extrusion. Briefly, phospholipids (hydrogenated soy phosphatidylcholine (HSPC), cholesterol (CHOL), and farnesol (FAR)) were combined in glass vials to achieve the lipid ratios detailed in the table above, specifically ratios of 85:15, 60:40, 80:15:5, 83:15:2, and 58:40:2. For formulations containing FAR, the component was added during the initial lipid film formation. Chloroform was removed via evaporation, and the resulting lipid films were stored in a desiccator.

The films were then hydrated with 10 mM HEPES buffer (pH 7.4) at 65° C. to achieve a final lipid concentration of 2.5 mg mL⁻¹. Hydrated films were vortexed and extruded through a 100 nm polycarbonate membrane ten times. Fluorescent liposomes were prepared with 0.1% (w/w) PE-LRB, while drug-loaded formulations included either FLU (Fluconazole) or MCZ (Miconazole) at low loading (1×) or high loading (5×) concentrations. For these drug-loaded liposomes, the active ingredients were solubilized in MeOH and added to the initial lipid mixture together with the other lipid components prior to film formation, ensuring proper incorporation during subsequent hydration and extrusion. Dynamic light scattering (DLS) was performed to determine the hydrodynamic diameter, polydispersity index (PDI), and zeta potential of the liposome formulations. A summary of the liposome formulations and physicochemical characterizations are provided in Table 1.

TABLE 1
Summary of liposome formulations and physicochemical characterization.
Hydrodynamic diameter, ζ-potential, and polydispersity index (PDI).
Data presented as mean ± SD (n = 9).

| | Hydrodynamic | | ζ-Potential |
|---|---|---|---|
| Formulation | Diameter (nm) | PDI | (mV) |
| 85:15 HSPC:CHOL | 113.63 ± 14.33 | 0.123 ± 0.042 | −3.67 ± 1.58 |
| 60:40 HSPC:CHOL | 118.30 ± 17.99 | 0.075 ± 0.018 | −10.83 ± 2.03 |
| 80:15:5 HSPC:CHOL:FAR | 114.47 ± 2.66 | 0.120 ± 0.022 | −11.25 ± 2.34 |
| 83:15:2 HSPC:CHOL:FAR | 115.33 ± 1.99 | 0.081 ± 0.026 | −8.92 ± 1.65 |
| 58:40:2 HSPC:CHOL:FAR | 130.38 ± 3.99 | 0.082 ± 0.012 | −5.93 ± 1.07 |
| 85:15 HSPC:CHOL 1X FLU | 112.5 ± 4.14 | 0.041 ± 0.005 | 12.1 ± 1.23 |
| 85:15 HSPC:CHOL 5X FLU | 113.1 ± 3.68 | 0.043 ± 0.001 | 5.3 ± 1.14 |
| 83:15:2 HSPC:CHOL:FAR 1X | 110.9 ± 8.92 | 0.031 ± 0.004 | −4.01 ± 0.71 |
| FLU | | | |
| 83:15:2 HSPC:CHOL:FAR 5X | 117 ± 2.23 | 0.051 ± 0.01 | 7.11 ± 0.75 |
| FLU | | | |
| 80:15:5 HSPC:CHOL:FAR 1X | 124.8 ± 5.53 | 0.058 ± 0.001 | −2.02 ± 0.4 |
| FLU | | | |
| 80:15:5 HSPC: CHOL:FAR 5X | 120.9 ± 1.99 | 0.078 ± 0.007 | −1.01 ± 0.81 |
| FLU | | | |
| 85:15 HSPC:CHOL 1X MCZ | 108.3 ± 4.23 | 0.104 ± 0.002 | −1.12 ± 1.12 |
| 85:15 HSPC:CHOL 5X MCZ | 126.4 ± 12.98 | 0.065 ± 0.002 | −4.11 ± 2.11 |
| 83:15:2 HSPC:CHOL:FAR 1X | 105.7 ± 9.35 | 0.021 ± 0.001 | 9.94 ± 2.32 |
| MCZ | | | |
| 83:15:2 HSPC:CHOL:FAR 5X | 113.4 ± 8.35 | 0.091 ± 0.003 | 3.21 ± 1.32 |
| MCZ | | | |
| 80:15:5 HSPC:CHOL:FAR 1X | 116.5 ± 6.24 | 0.111 ± 0.021 | 6.51 ± 0.96 |
| MCZ | | | |
| 80:15:5 HSPC:CHOL:FAR 5X | 120.4 ± 11.85 | 0.051 ± 0.009 | 2.51 ± 1.17 |
| MCZ | | | |

In one embodiment, the present disclosure provides measuring the hydrodynamic diameter of the liposome formulations using dynamic light scattering (DLS). Each liposome of the plurality of liposomes has a hydrodynamic diameter in the range of about 50 nm to about 200 nm in diameter. In other embodiments, each liposome of the plurality of liposomes has a hydrodynamic diameter in the range of about 100 nm to about 150 nm in diameter. In certain embodiments, the liposome formulations exhibited hydrodynamic diameters ranging from approximately 113 nm to 132 nm depending on the specific molar ratio of lipid components.

According to some embodiments, the liposome formulation had a molar ratio of HSPC:CHOL of 85:15 without farnesol. The 85:15 HSPC:CHOL formulation exhibited a hydrodynamic diameter of approximately 113.63±14.33 nm. In other embodiments, the liposome formulation had a molar ratio of HSPC:CHOL of 60:40 without farnesol. The 60:40 HSPC:CHOL formulation exhibited a hydrodynamic diameter of approximately 118.30±17.99 nm.

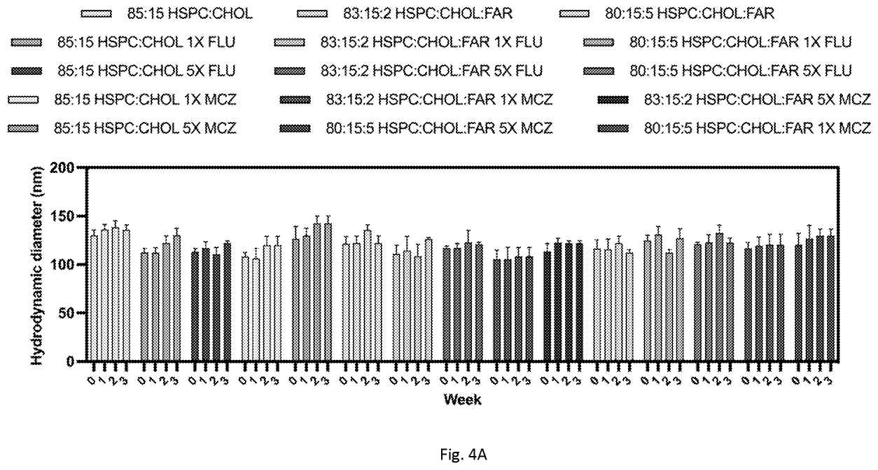

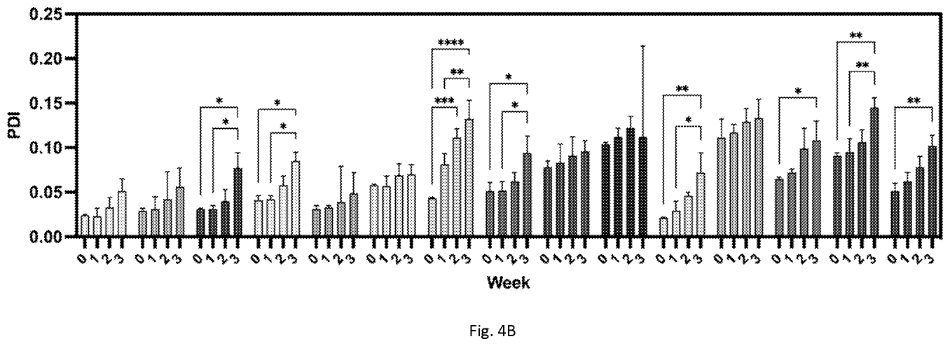

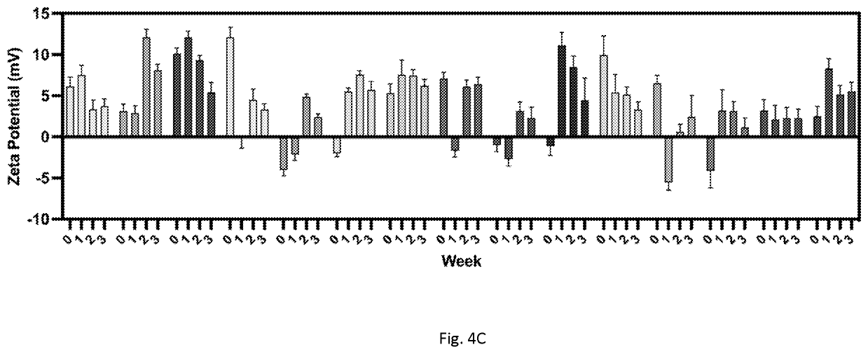

As shown in FIG. 4A, the molar ratio of HSPC:CHOL:FAR may be in the range of 78-85:15-40:2-5, e.g., for DOTAP coated liposomes. In certain embodiments, the liposome formulation had a molar ratio of HSPC:CHOL:FAR of 83:15:2. The 83:15:2 HSPC:CHOL:FAR formulation exhibited a hydrodynamic diameter of approximately 115.33±1.99 nm. In some embodiments, the molar ratio of HSPC:CHOL:FAR was 80:15:5. The 80:15:5 HSPC:CHOL:FAR formulation exhibited a hydrodynamic diameter of approximately 114.47±2.66 nm. In certain embodiments, the liposome formulation had a molar ratio of HSPC:CHOL:FAR of 58:40:2. The 58:40:2 HSPC:CHOL:FAR formulation exhibited a hydrodynamic diameter of approximately 130.38±3.99 nm.

As shown in FIG. 4B, the zeta potential of the liposome formulations was assessed to characterize the surface charge of the liposomes. The liposomes have a zeta potential ranging from approximately −28 mV to +30 mV depending on formulation and coating. The 85:15 HSPC:CHOL formulation exhibited a zeta potential of approximately −3.67±1.58 mV. The 60:40 HSPC:CHOL formulation exhibited a zeta potential of approximately −10.83±2.03 mV. The 83:15:2 HSPC:CHOL:FAR formulation exhibited a zeta potential of approximately −8.92±1.65 mV. The 80:15:5 HSPC:CHOL:FAR formulation exhibited a zeta potential of approximately −11.25±2.34 mV. The 58:40:2 HSPC:CHOL:FAR formulation exhibited a zeta potential of approximately −5.93±1.07 mV.

As shown in FIG. 4C, the polydispersity index (PDI) of each formulation was measured to assess the uniformity of liposome size distribution. The liposomes had a polydispersity index (PDI) ranging from approximately 0.02 to 0.22 depending on formulation. The 85:15 HSPC:CHOL formulation exhibited a PDI of approximately 0.123±0.042. The 60:40 HSPC:CHOL formulation exhibited a PDI of approximately 0.075±0.018. The 83:15:2 HSPC:CHOL:FAR formulation exhibited a PDI of approximately 0.081±0.026. The 80:15:5 HSPC:CHOL:FAR formulation exhibited a PDI of approximately 0.120±0.022. The 58:40:2 HSPC:CHOL:FAR formulation exhibited a PDI of approximately 0.082±0.012. The low PDI values indicated that the extrusion process through the 100 nm polycarbonate membrane produced uniformly sized liposomes across all formulations tested.

### Example II

Neutralizing the Damaging Effects of Candidalysin Upon its Release from Hyphal Cells

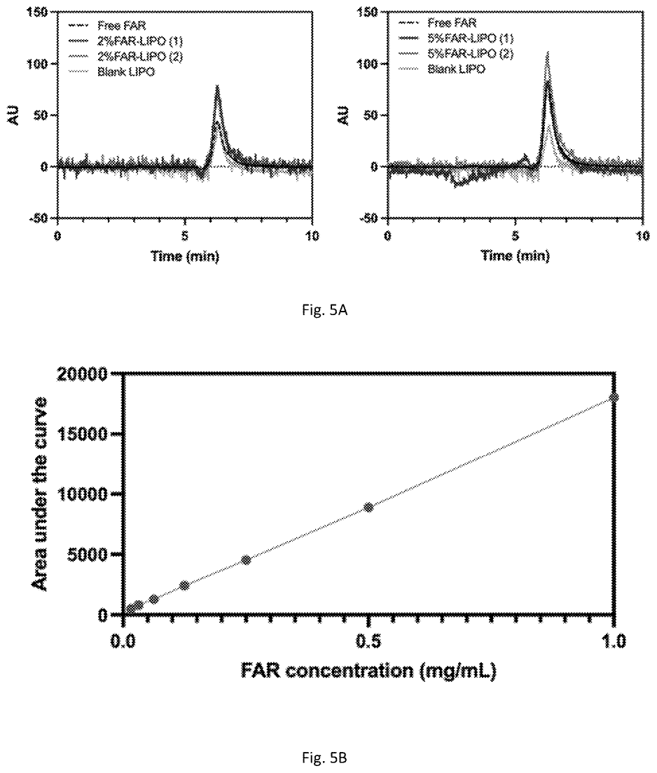

In one embodiment, the present disclosure investigates neutralizing the damaging effects of candidalysin upon its release from hyphal cells. In one embodiment, the present disclosure provides quantitative analysis of farnesol (FAR) encapsulation as performed via high-performance liquid chromatography (HPLC). FIG. 5A shows representative chromatograms of free farnesol, 2% FAR-loaded liposomes (FAR-LIPO), 5% FAR-LIPO, and blank liposome controls. All FAR-containing formulations exhibited a characteristic absorbance peak at a retention time of approximately 6.2 minutes. The blank liposome controls did show a peak at 6.2 min, which were used as the baseline, and this baseline was subtracted from the farnesol-containing peaks to calculate farnesol entrapment efficiency, confirming that the additional signal corresponds to farnesol. The data representing the mean and standard deviation (n=3) for 2% and 5% FAR-loaded liposomes (FAR-LIPO) and entrapment efficiencies are provided in Table 2.

TABLE 2
Data represent the mean and standard deviation (n = 3) for 2% and 5% FAR-
loaded liposomes (FAR-LIPO). Entrapment efficiency (%) was calculated
based on the ratio of encapsulated FAR to the initial drug input, as determined
by HPLC analysis. (e.g., shown in FIG. 5.)

| Formulation | Farnesol entrapment efficiency |
|---|---|
| 83:15:2 HSPC:CHOL:FAR | 80.67% ± 0.134% |
| 80:15:5 HSPC:CHOL:FAR | 72.80% ± 0.104% |

The method for FIG. 5 of the present disclosure provides that farnesol entrapment efficiency was assessed using high-performance liquid chromatography (HPLC). Liposome samples were taken directly from the stock suspension and sterile-filtered prior to analysis without prior disruption. Chromatographic separation was performed using an isocratic mobile phase consisting of methanol and water (80:20, v/v) at a flow rate of 0.6 mL/min. A 5 μL injection volume was used, and farnesol was detected at 210 nm. The high methanol content of the mobile phase facilitated liposome disruption during analysis, allowing quantification of the encapsulated farnesol. Entrapment efficiency was determined by comparing the measured farnesol concentration to the initial amount of farnesol used during liposome preparation.

In one embodiment, as shown in FIGS. 5A-5B, the HPLC analysis was performed using an isocratic mobile phase consisting of methanol and water at a ratio of 80:20 (v/v) at a flow rate of 0.6 mL/min. A 5 μL injection volume was used, and farnesol was detected at 210 nm. The high methanol content of the mobile phase facilitated liposome disruption during analysis, allowing quantification of the encapsulated farnesol. Liposome samples were taken directly from the stock suspension and sterile-filtered prior to analysis without prior disruption.

As further shown in FIG. 5B, a calibration curve showed the relationship between FAR concentration and the integrated area under the curve (AUC). The calibration curve was used to determine the farnesol concentration in the liposome formulations based on the measured AUC values from the chromatograms. Entrapment efficiency was determined by comparing the measured farnesol concentration to the initial amount of farnesol used during liposome preparation.

The farnesol entrapment efficiency was approximately 80.67%±0.134% for 2% FAR-loaded liposomes. The farnesol entrapment efficiency was approximately 72.80%±0.104% for 5% FAR-loaded liposomes. The 83:15:2 HSPC:CHOL:FAR formulation, which corresponded to the 2% FAR-loaded liposomes, exhibited the higher entrapment efficiency. The 80:15:5 HSPC:CHOL:FAR formulation, which corresponded to the 5% FAR-loaded liposomes, exhibited a slightly lower entrapment efficiency. The entrapment efficiency data indicated that farnesol may be successfully incorporated into the lipid bilayer of the liposomes during the thin-film hydration fabrication process.

According to one aspect, the present disclosure provides methods and results for antihyphal liposomes incubated with C. albicans SC5314.

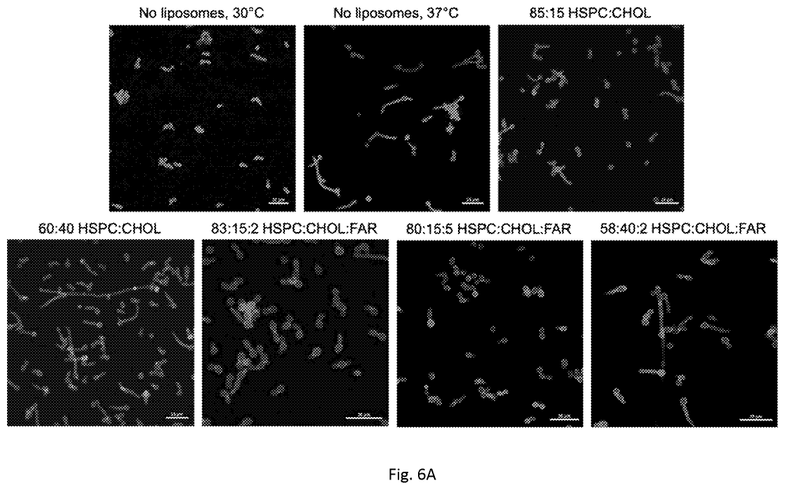

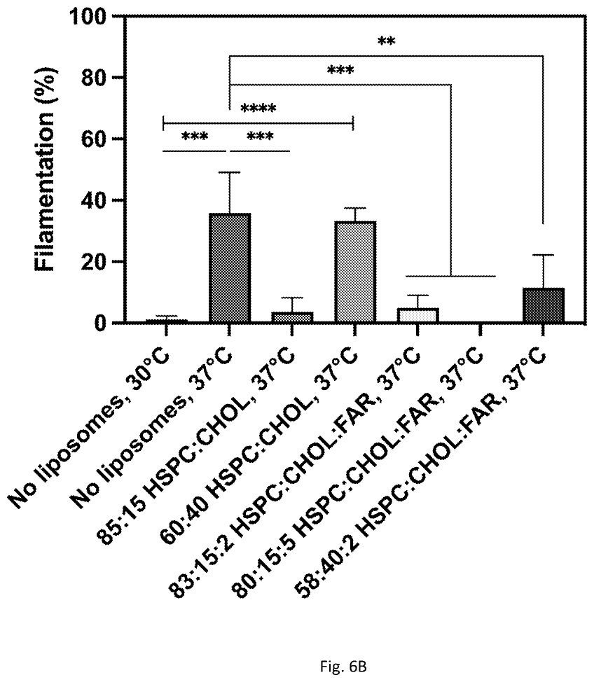

The method for FIG. 6 of the present disclosure provides that C. albicans cells were prepared and incubated with antihyphal liposomes. Briefly, overnight culture of commonly used reference strain (C. albicans SC5314) was incubated at 37° C. and 150 rpm for 16 h. Then, 20-fold dilutions of the C. albicans overnight cultures were prepared in YNB-AS media and incubated at 37° C. and 150 rpm in a 1.4:0.1:0.5 (YNB-AS:liposomes: Candida culture, (v/v)) ratio for 2-3 h. Suspensions were then stored on ice, and, immediately prior to imaging, C. albicans cells were stained with 1% (v/v) CFW stain in 0.1 M NaOH. Stained C. albicans samples (10 μL) were transferred to glass slides, covered with a glass cover slip, and imaged using a 20× objective.

Yeast and hyphal morphologies of C. albicans were quantified using an image segmentation and automated classification workflow. Confocal microscopy images were first segmented using the ‘Segment Anything for Microscopy’ plugin in the Napari environment with the ‘Light Microscopy’ model. Automatic segmentation was performed to generate object masks, and incorrect or missed segmentations were manually corrected using prompt-based annotations (box or freehand tools). The finalized segmentation masks were exported and processed using a custom Python script to count individual cells and classify morphologies. Classification was based on object aspect ratio, where cells with an aspect ratio ≤3.0 were categorized as yeast and those with an aspect ratio >3.0 were categorized as pseudohyphae or hyphae. The script analyzed each mask image and generated a CSV file containing total cell counts and the relative proportions of each morphology.

As shown in FIG. 6A, confocal imaging of antihyphal liposomes incubated with C. albicans SC5314 demonstrates liposome interaction with yeast and hyphal morphologies. The liposome formulation may include a fluorescent dye for imaging purposes. In some embodiments, the fluorescent dye is 0.1% (w/w) 1,2-distearoyl-sn-glycero-3-phosphoethanolamine-N-(Lissamine rhodamine B sulfonyl) (LRB) incorporated into the lipid bilayer. The LRB fluorescent label allows visualization of liposome localization relative to fungal cells during confocal microscopy. The images depict the changes in morphology in C. albicans.

As further shown in FIG. 6A, the representative images showed liposome interaction with both yeast and hyphal morphologies of C. albicans SC5314. The C. albicans cells were prepared from an overnight culture of the commonly used reference strain SC5314 incubated at 37° C. and 150 rpm for 16 hours. Twenty-fold dilutions of the C. albicans overnight cultures were prepared in YNB-AS media and incubated at 37° C. and 150 rpm in a 1.4:0.1:0.5 (YNB-AS:liposomes: Candida culture, v/v) ratio for 2-3 hours. Prior to imaging, C. albicans cells were stained with 1% (v/v) calcofluor white (CFW) stain in 0.1 M NaOH to visualize fungal cell walls.

As further shown in FIG. 6A, confocal microscopy images illustrated that treatment with antihyphal liposomes induces a noticeable change in C. albicans morphology. The antihyphal liposomes revealed alterations in filamentation, with treated cells exhibiting reduced hyphal formation compared to untreated controls, confirming the antihyphal activity of the liposome formulations.

As shown in FIG. 6B, quantification of yeast versus hyphal cell counts upon liposome treatment demonstrated that the liposomes preferentially interact with hyphal cells compared to yeast cells of C. albicans. The yeast and hyphal morphologies of C. albicans were quantified using an image segmentation and automated classification workflow. Confocal microscopy images were segmented using the “Segment Anything for Microscopy” plugin in the Napari environment with the “Light Microscopy” model.

As shown in FIG. 6B, classification of fungal cell morphology was based on object aspect ratio. Cells with an aspect ratio of 3.0 or less were categorized as yeast, and cells with an aspect ratio greater than 3.0 were categorized as pseudohyphae or hyphae. The segmentation masks were processed using a custom Python script to count individual cells and classify morphologies. The script analyzed each mask image and generated data containing total cell counts and the relative proportions of each morphology.

As further shown in FIG. 6B, the quantification data indicated that the antihyphal liposomes exhibit preferential interaction with hyphal cells. The preferential interaction of liposomes with hyphal cells enhanced the delivery of farnesol and other therapeutic agents to the pathogenic hyphal form of C. albicans. The differential interaction between liposomes and fungal cell morphologies contributed to the antihyphal activity of the liposome composition by targeting the hyphal cells that are associated with virulence and tissue invasion.

In one embodiment, the present disclosure provides evaluating of the antifungal activity of drug-loaded liposomes by measuring normalized planktonic growth of C. albicans SC5314 following exposure to free drug, blank liposomes, and antihyphal liposomes loaded with fluconazole (FLU). Each liposome of the plurality of liposomes further encapsulated an antifungal therapeutic selected from the group consisting of an azole, a polyene, and an echinocandin. In some embodiments, the antifungal therapeutic was an azole selected from the group consisting of posaconazole, fluconazole, voriconazole, and miconazole.

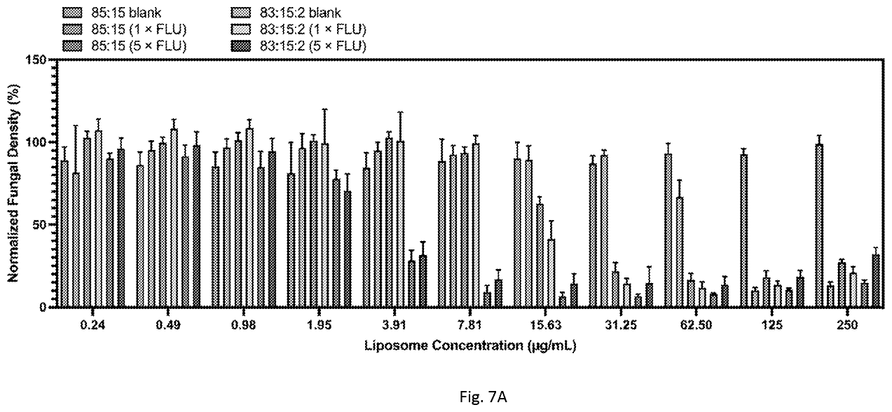

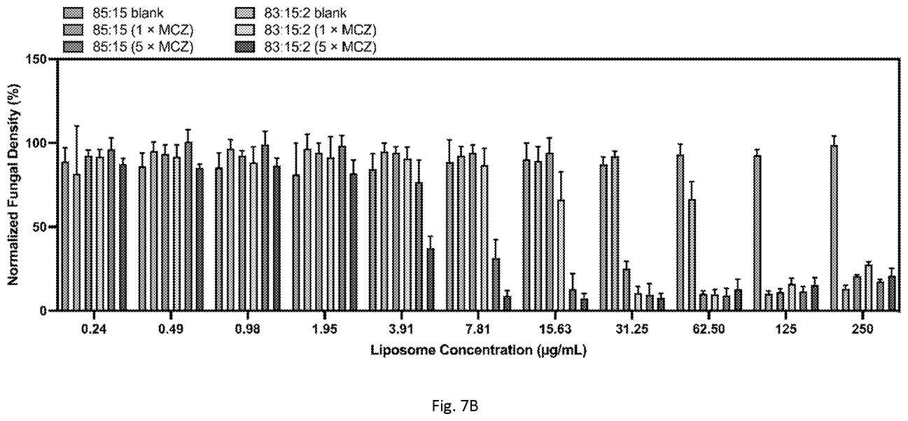

As shown in FIG. 7A, drug-loaded liposomes was prepared by forming the lipid film directly over a drug film before hydration, ensuring proper incorporation of antifungal therapeutics. The active ingredients were solubilized in methanol and added to the initial lipid mixture together with the other lipid components prior to film formation. The lipid film was then formed directly over the drug film, and subsequent hydration and extrusion incorporate the antifungal therapeutic into the liposome structure.

As further shown in FIG. 7A, the normalized planktonic growth data demonstrated the antifungal activity of fluconazole-loaded liposomes against C. albicans SC5314. The 85:15 HSPC:CHOL formulation loaded with 1×FLU exhibited a minimum inhibitory concentration for 50% growth inhibition (MIC₅₀) at a theoretical drug concentration of 0.048 μg/mL. The 85:15 HSPC:CHOL formulation loaded with 5×FLU exhibited an MIC₅₀ at a theoretical drug concentration of 0.061 μg/mL. The 83:15:2 HSPC:CHOL:FAR formulation loaded with 1×FLU exhibited an MIC₅₀ at a theoretical drug concentration of 0.048 μg/mL. The 83:15:2 HSPC:CHOL:FAR formulation loaded with 5×FLU exhibited an MIC₅₀ at a theoretical drug concentration of 0.061 μg/mL.

As shown in FIG. 7B, the antifungal activity of antihyphal liposomes loaded with miconazole (MCZ) was evaluated against C. albicans SC5314. The antifungal therapeutic miconazole (MCZ) may be encapsulated in the liposomes at low loading (1×) or high loading (5×) concentrations. The 85:15 HSPC:CHOL formulation loaded with 1×MCZ exhibited an MIC₅₀ at a theoretical drug concentration of 0.097 μg/mL. The 85:15 HSPC:CHOL formulation loaded with 5×MCZ exhibited an MIC₅₀ at a theoretical drug concentration of 0.121 μg/mL.

As further shown in FIG. 7B, the 83:15:2 HSPC:CHOL:FAR formulation loaded with 1×MCZ exhibited an MIC₅₀ at a theoretical drug concentration of 0.097 μg/mL. The 83:15:2 HSPC:CHOL:FAR formulation loaded with 5×MCZ exhibited an MIC₅₀ at a theoretical drug concentration of 0.061 μg/mL. The blank 83:15:2 HSPC:CHOL:FAR formulation without drug loading exhibited an MIC₅₀ at a liposome concentration of 62.50 μg/mL, indicating that the farnesol-containing liposomes exhibit antifungal activity independent of encapsulated drug.

As further shown in FIGS. 7A-7B, the miconazole-loaded liposomes exhibited lower MIC₅₀ values compared to the fluconazole-loaded liposomes at equivalent loading concentrations. Miconazole has a higher log P value of 5.3 compared to fluconazole with a log P value of 0.5, indicating that miconazole is more hydrophobic and may be more efficiently incorporated into the lipid bilayer of the liposomes. The drug-loaded liposomes maintained hydrodynamic diameters within the range of approximately 105 nm to 126 nm, indicating that drug loading did not substantially alter the physical characteristics of the liposomes.

A summary of the experimental conditions such as the various liposome formulations and MIC₅₀ for FIGS. 7A and 7B are provided in Table 3.

TABLE 3
Summary of the experimental conditions for FIGS. 7A and 7B.

| | MIC₅₀ (Theoretical |
|---|---|
| Formulation | drug concentration) |
| 85:15 HSPC:CHOL blank | — |
| 85:15 HSPC:CHOL 1X FLU | 0.048 μg/mL |
| 85:15 HSPC:CHOL 5X FLU | 0.061 μg/mL |
| 83:15:2 HSPC:CHOL:FAR blank | 62.50 μg/mL |
| | (liposome concentration) |
| 83:15:2 HSPC:CHOL:FAR 1X FLU | 0.048 μg/mL |
| 83:15:2 HSPC:CHOL:FAR 5X FLU | 0.061 μg/mL |
| 85:15 HSPC:CHOL 1X MCZ | 0.097 μg/mL |
| 85:15 HSPC:CHOL 5X MCZ | 0.121 μg/mL |
| 83:15:2 HSPC:CHOL:FAR 1X MCZ | 0.097 μg/mL |
| 83:15:2 HSPC:CHOL:FAR 5X MCZ | 0.061 μg/mL |

In one embodiment, the antifungal activity of free drugs against planktonic C. albicans SC5314 was evaluated by measuring normalized growth after exposure to free posaconazole, fluconazole, and voriconazole. The minimum inhibitory concentration (MIC) of free drug formulations against C. albicans was determined using a broth microdilution assay adapted from the European Committee on Antimicrobial Susceptibility Testing (EUCAST) protocol for yeasts.

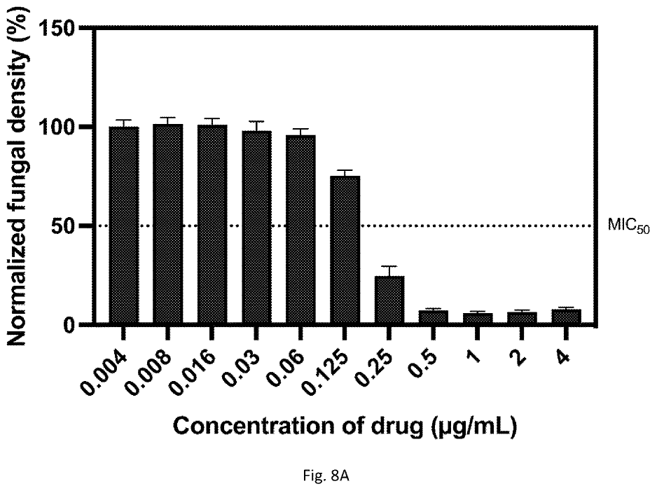

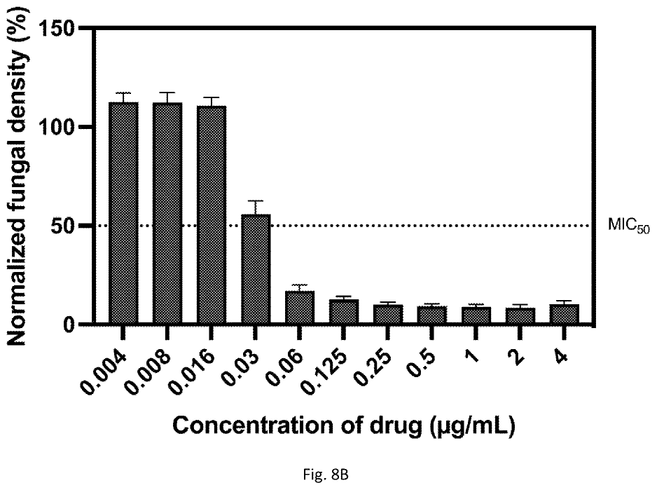

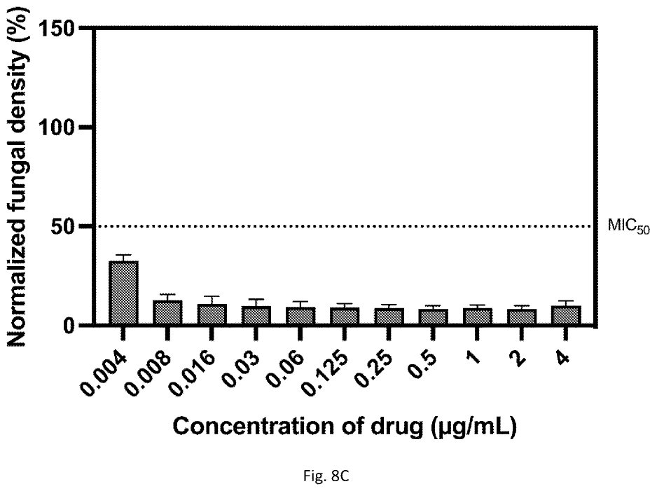

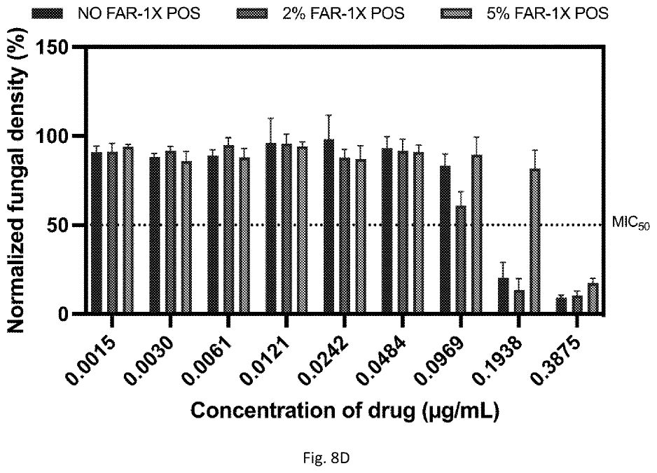

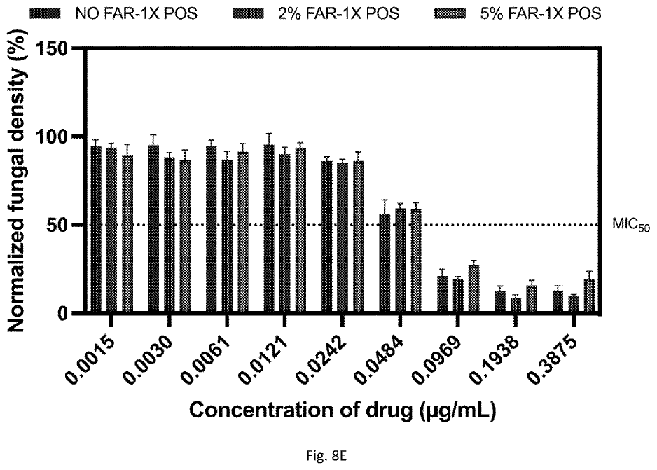

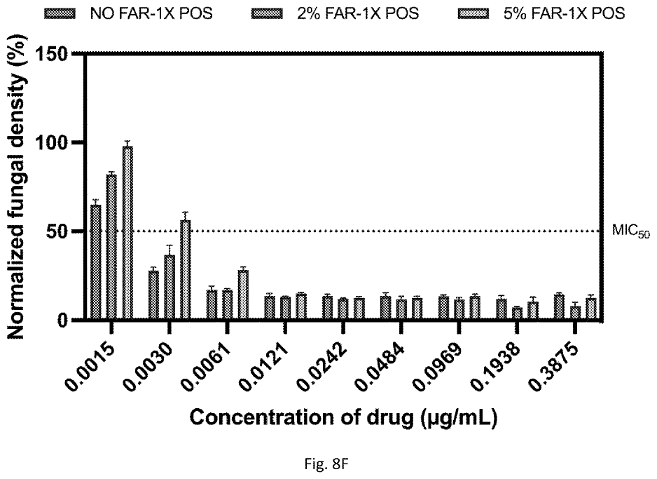

As shown in FIG. 8A, free posaconazole exhibited an MIC₅₀ greater than 0.25 μg/mL against C. albicans SC5314. Posaconazole has a molecular weight of 700.7 g/mol and a log P value of 5.5, indicating high hydrophobicity. The normalized growth data demonstrated that posaconazole requires relatively higher concentrations to achieve 50% growth inhibition compared to the other azole antifungals tested.

As shown in FIG. 8B, free fluconazole exhibited an MIC₅₀ of 0.06 μg/mL against C. albicans SC5314. Fluconazole has a molecular weight of 306.3 g/mol and a log P value of 0.5, indicating relatively lower hydrophobicity compared to posaconazole. The normalized growth measurements showed dose-dependent inhibition of planktonic C. albicans growth with increasing fluconazole concentrations.

As further shown in FIG. 8C, free voriconazole exhibited an MIC₅₀ of less than 0.004 μg/mL against C. albicans SC5314. Voriconazole has a molecular weight of 349.3 g/mol and a log P value of 1.82. The voriconazole data demonstrated that voriconazole exhibits the highest potency among the three azole antifungals tested, achieving 50% growth inhibition at concentrations below the lowest tested concentration.

As shown in FIGS. 8D-8F, the antifungal activity of blank liposomes and antihyphal liposomes loaded with posaconazole, fluconazole, and voriconazole was evaluated against planktonic C. albicans SC5314. The liposome formulations tested included 85:15 HSPC:CHOL, 83:15:2 HSPC:CHOL:FAR, and 80:15:5 HSPC:CHOL:FAR, each loaded with 1× drug concentration corresponding to 0.31% (w/w) drug loading.

As shown in FIG. 8D, posaconazole-loaded liposomes exhibited MIC₅₀ values greater than 0.1938 μg/mL for the 83:15:2 HSPC:CHOL:FAR formulation and greater than 0.3875 μg/mL for the 80:15:5 HSPC:CHOL:FAR formulation. The 85:15 HSPC:CHOL formulation loaded with 1× posaconazole exhibited an MIC₅₀ greater than 0.1938 μg/mL. The posaconazole-loaded liposome formulations exhibited similar activity profiles to free posaconazole, with relatively higher concentrations required for growth inhibition.

As shown in FIG. 8E, fluconazole-loaded liposomes exhibited MIC₅₀ values of 0.0484 μg/mL for the 83:15:2 HSPC:CHOL:FAR formulation, 0.0484 μg/mL for the 80:15:5 HSPC:CHOL:FAR formulation, and 0.0484 μg/mL for the 85:15 HSPC:CHOL formulation. The fluconazole-loaded liposome formulations exhibited comparable MIC₅₀ values to free fluconazole, indicating that encapsulation in liposomes maintains the antifungal activity of fluconazole.

As further shown in FIG. 8F, voriconazole-loaded liposomes exhibited MIC₅₀ values of 0.003 μg/mL for the 83:15:2 HSPC:CHOL:FAR formulation, 0.003 μg/mL for the 80:15:5 HSPC:CHOL:FAR formulation, and 0.003 μg/mL for the 85:15 HSPC:CHOL formulation. The voriconazole-loaded liposome formulations exhibited MIC₅₀ values comparable to free voriconazole, demonstrating that the liposome encapsulation does not diminish the potency of voriconazole against C. albicans.

The comparative data from FIGS. 8A-8F demonstrated that the farnesol-containing liposome formulations (83:15:2 HSPC:CHOL:FAR and 80:15:5 HSPC:CHOL:FAR) exhibited antifungal activity comparable to the blank 85:15 HSPC:CHOL formulation when loaded with equivalent drug concentrations. The incorporation of farnesol into the lipid bilayer did not interfere with the antifungal activity of the encapsulated azole drugs. The liposome formulations provided a delivery vehicle for azole antifungals while simultaneously delivering farnesol to inhibit hyphal formation. The drugs and experimental conditions for FIGS. 8A to 8B have been provided in Table 4 and Table 5.

TABLE 4
Summary of drugs used for FIGS. 8A to 8F.

| Formulation | MIC₅₀ (Drug concentration) |
|---|---|
| Fluconazole | 0.06 μg/mL |
| Posaconazole | >0.25 μg/mL |
| Voriconazole (VOR) | <0.004 μg/mL |

TABLE 5
Summary of the experimental conditions for FIGS. 8A to 8F.

| Formulation | MIC₅₀ (Theoretical drug concentration) |
|---|---|
| 83:15:2 HSPC:CHOL:FAR 1X POS | >0.1938 μg/mL |
| 80:15:5 HSPC:CHOL:FAR 1X POS | >0.3875 μg/mL |
| 85:15 HSPC:CHOL 1X POS | >0.1938 μg/mL |
| 83:15:2 HSPC:CHOL:FAR 1X FLU | 0.0484 μg/mL |
| 80:15:5 HSPC:CHOL:FAR 1X FLU | 0.0484 μg/mL |
| 85:15 HSPC:CHOL 1X FLU | 0.0484 μg/mL |
| 83:15:2 HSPC:CHOL:FAR 1X VOR | 0.003 μg/mL |
| 80:15:5 HSPC:CHOL:FAR 1X VOR | 0.003 μg/mL |
| 85:15 HSPC:CHOL 1X VOR | 0.003 μg/mL |

The method for FIGS. 8A and 8B of the present disclosure provides that minimum inhibitory concentration (MIC) of free drug liposome formulations against C. albicans was determined using a broth microdilution assay adapted from the European Committee on Antimicrobial Susceptibility Testing (EUCAST) protocol for yeasts. Liposome suspensions were serially diluted two-fold in HEPES buffer to generate a range of concentrations and subsequently mixed 1:1 with double-strength RPMI 1640 medium supplemented with 2% glucose (RPMI-2% G) to obtain working solutions at 2× the desired final concentrations. Free drug solutions were prepared and diluted in the same manner for comparison. C. albicans inoculum was prepared from fresh agar cultures and standardized to a 0.5 McFarland turbidity standard (OD₅₃₀=0.11-0.14), corresponding to approximately 1-5×10⁶ CFU/mL, followed by a 1:10 dilution to obtain a working inoculum of 1-5×10⁵ CFU/mL. In 96-well microdilution plates, 100 μL of free drug or each liposome dilution was combined with 100 μL of the yeast suspension to achieve a final volume of 200 μL per well and the desired test concentrations. Plates included positive growth controls, formulation background controls, and sterility controls. Following incubation at 37° C. for 24 h without agitation, fungal growth was assessed by measuring absorbance at 530 nm. The MIC₉₀ and MIC₅₀ were defined as the lowest concentrations resulting in ≥50% and ≥90% inhibition of growth, respectively, relative to the drug-free growth control.

In one embodiment, present disclosure provides assessment of the inhibition of biofilm biomass by crystal violet staining upon exposure to blank and drug-loaded antihyphal liposomes. The liposome composition inhibits biofilm formation in C. albicans as measured by crystal violet staining assay. The minimum biofilm inhibitory concentration (MBIC) of liposome formulations against C. albicans was determined using a biofilm inhibition assay adapted from previously reported protocols.

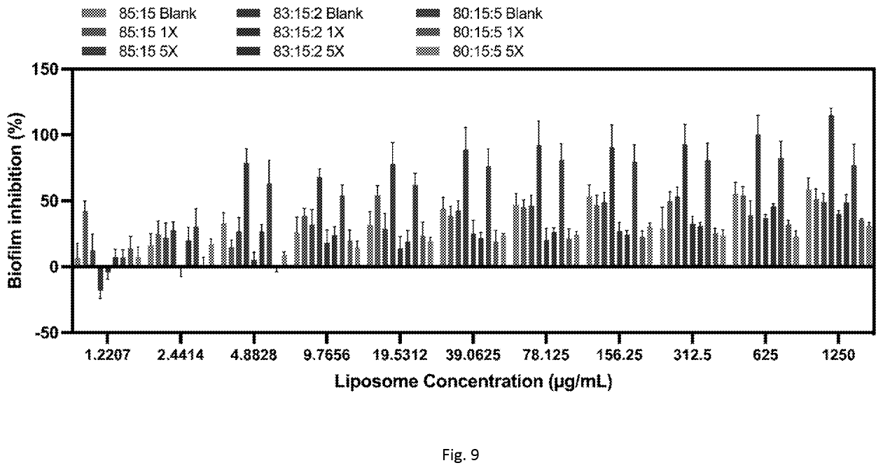

As shown in FIG. 9, overnight cultures of C. albicans SC5314 were grown in YPD at 37° C. for approximately 16 hours, pelleted, and resuspended in RPMI 1640 medium supplemented with additional glucose to obtain a cell density of approximately 1×10⁶ CFU/mL. For inhibition assays, yeast suspensions were combined with liposome treatments in 96-well plates coated with fetal bovine serum. Each well contained 270 μL of the standardized cell suspension and 30 μL of liposome solution, resulting in a final volume of 300 μL per well. Plates were sealed and incubated at 37° C. with shaking at 75 rpm for 24 hours to allow biofilm formation in the presence of treatments.

As further shown in FIG. 9, biofilm biomass was quantified using a crystal violet staining assay. Biofilms were fixed with methanol, stained with 0.1% crystal violet, washed with PBS, and the bound dye was solubilized with ethanol or methanol. Absorbance was measured at 540 nm to quantify biofilm biomass. The MBIC₅₀ was defined as the lowest concentration resulting in 50% or greater inhibition of biofilm formation relative to the drug-free growth control.

The 85:15 HSPC:CHOL blank formulation exhibited an MBIC₅₀ at a liposome concentration of 39.06 μg/mL. The 85:15 HSPC:CHOL formulation loaded with 1× drug (0.31% w/w) exhibited an MBIC₅₀ at a liposome concentration of 19.53 μg/mL, corresponding to a theoretical drug concentration of 0.061 μg/mL. The 85:15 HSPC:CHOL formulation loaded with 5× drug (1.55% w/w) exhibited an MBIC₅₀ at a liposome concentration of 39.06 μg/mL, corresponding to a theoretical drug concentration of 0.605 μg/mL.

As shown in FIG. 9, the 83:15:2 HSPC:CHOL:FAR blank formulation exhibited an MBIC₅₀ at a liposome concentration of 39.06 μg/mL. The 83:15:2 HSPC:CHOL:FAR formulation loaded with 1× drug exhibited an MBIC₅₀ at a liposome concentration of 625 μg/mL, corresponding to a theoretical drug concentration of 1.93 μg/mL. The 83:15:2 HSPC:CHOL:FAR formulation loaded with 5× drug exhibited an MBIC₅₀ at a liposome concentration of 625 μg/mL, corresponding to a theoretical drug concentration of 9.63 μg/mL.

As further shown in FIG. 9, the 80:15:5 HSPC:CHOL:FAR blank formulation exhibited an MBIC₅₀ at a liposome concentration of 9.77 μg/mL. The 80:15:5 HSPC:CHOL:FAR formulation loaded with either 1× or 5× fluconazole did not reach the MBIC₅₀, as even at the highest tested liposome concentrations, 50% inhibition of C. albicans biofilm formation was not achieved.

The biofilm inhibition results indicated that both liposome composition and drug loading influence antifungal activity. Incorporation of farnesol into the lipid formulation significantly enhanced biofilm inhibition, as demonstrated by the 80:15:5 HSPC:CHOL:FAR blank formulation, which exhibited the lowest MBIC₅₀ value (9.77 μg/mL) compared to the blank 85:15 HSPC:CHOL formulation (39.06 μg/mL). This result indicates that farnesol alone contributes substantially to antibiofilm activity. Fluconazole loading further improved activity in the 85:15 formulation at low loading, where the 1×(0.31%) formulation reduced the MBIC₅₀ to 19.53 μg/mL. However, increasing drug loading to 5× did not provide additional improvement, and in the 83:15:2 formulation fluconazole loading substantially reduced activity (MBIC₅₀=625 μg/mL). Overall, the data demonstrate that liposome formulations without farnesol exhibit measurable antibiofilm activity, as shown by the blank 85:15 HSPC:CHOL formulation. However, incorporation of farnesol enhanced this activity, with the 80:15:5 HSPC:CHOL:FAR blank formulation showing the greatest biofilm inhibition. Notably, loading the liposomes with fluconazole did not further improve antibiofilm efficacy and, in several cases, resulted in reduced activity compared to the corresponding blank formulations. These results indicate that farnesol-containing liposomes alone are sufficient to drive antibiofilm activity, and drug loading is not required to achieve this effect. The experimental conditions for FIG. 9 is summarized in Table 6.

TABLE 6
Summary of the experimental condition for FIG. 9.

| | Theoretical | MBIC₅₀ | Theoretical |
|---|---|---|---|
| Formulation | drug | Liposome | Drug |
| (HSPC:CHOL:FAR) | loading | (μg/mL) | (μg/mL) |
| Free FLU | — | — | 2 |
| 85:15 | Blank | 39.06 | — |
| 85:15 | 1×(0.31%) | 19.53 | 0.061 |
| 85:15 | 5×(1.55%) | 39.06 | 0.605 |
| 83:15:2 | Blank | 39.06 | — |
| 83:15:2 | 1×(0.31%) | 625 | 1.93 |
| 83:15:2 | 5×(1.55%) | 625 | 9.68 |
| 80:15:5 | Blank | 9.77 | — |
| 80:15:5 | 1×(0.31%) | — | — |

The method for FIG. 9 of the present disclosure provides that minimum biofilm inhibitory concentration (MBIC) of liposome formulations against C. albicans was determined using a biofilm inhibition assay adapted from previously reported protocols. Overnight cultures of C. albicans SC5314 were grown in YPD at 37° C. for approximately 16 h, pelleted, and resuspended in RPMI 1640 medium supplemented with additional glucose to obtain a cell density of approximately 1×10⁶ CFU/mL (OD₅₃₀≈0.5-0.6). For inhibition assays, yeast suspensions were combined with liposome treatments in a 96-well plates coated with fetal bovine serum. Each well contained 180 μL of the standardized cell suspension and 20 μL of liposome solution, resulting in a final volume of 200 μL per well. Positive growth controls contained cells with buffer used for liposome dilution, while negative controls contained medium only. Plates were sealed and incubated at 37° C. with shaking (75 rpm) for 24 h to allow biofilm formation in the presence of treatments. Biofilm biomass was quantified using a crystal violet staining assay, in which biofilms were fixed with methanol, stained with 0.1% crystal violet, washed with PBS, and the bound dye was solubilized with ethanol or methanol. Absorbance was measured at 540 nm to quantify biofilm biomass.

The liposome composition disrupted pre-existing fungal biofilms when administered to a subject. The combination of farnesol and encapsulated antifungal therapeutics in the liposome formulations provided enhanced biofilm inhibition by targeting both hyphal formation and fungal cell viability. The biofilm inhibition activity of the liposome composition reduced the pathogenicity of C. albicans infections by preventing the formation of biofilm structures that contribute to antifungal drug resistance and treatment failure.

In another embodiment, the present disclosure provides evaluating the cytocompatibility of antihyphal liposomes by measuring the viability of vaginal epithelial cells upon treatment with blank and drug-loaded liposomes. The liposome composition showed no cytotoxicity against vaginal epithelial cells as demonstrated by the cell viability data. The cytocompatibility evaluation supported the biocompatibility profile of the antihyphal liposome formulations for potential therapeutic applications.

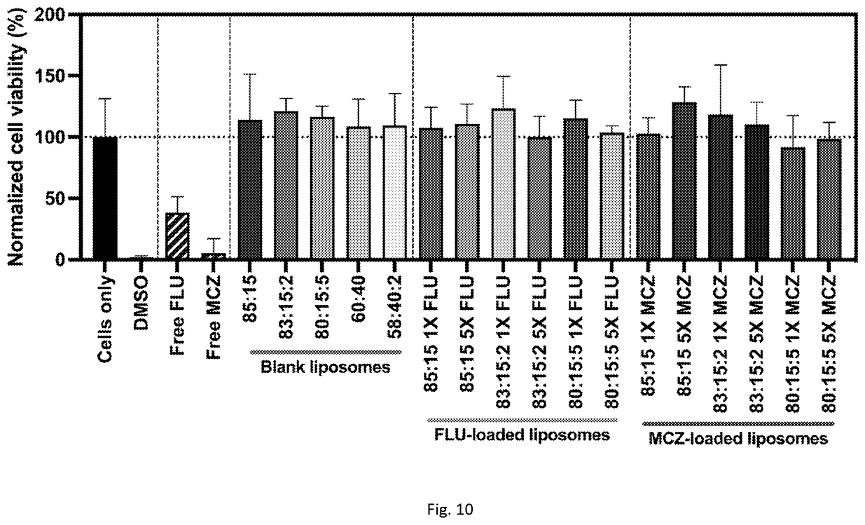

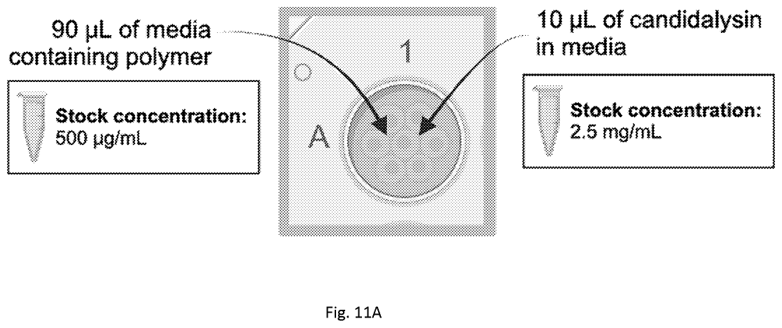

As shown in FIG. 10, the cytotoxicity of liposomes was evaluated against vaginal epithelial cells using a CCK-8 assay. Vaginal epithelial cells were cultured in keratinocyte SFM supplemented with 0.1 ng/mL recombinant epidermal growth factor (rEGF), 0.05 mg/mL bovine pituitary extract (BPE), and calcium chloride at 44.1 mg/L at 37° C. and 5% CO₂ until 80% confluent. Cells were seeded in tissue culture-treated 96-well plates at a density of 10,000 cells/cm², corresponding to approximately 3,200 cells per well, and incubated for 24 hours at 37° C. and 5% CO₂.

As further shown in FIG. 10, cells were treated with serial dilutions of each liposome formulation with a highest concentration of 250 μg/mL for approximately 16 hours under the same culture conditions. Following treatment, the media was replaced with fresh media containing 10% (v/v) CCK-8 reagent, and plates were incubated for an additional 2 hours at 37° C. and 5% CO₂ before measuring absorbance at 450 nm. Positive controls consisting of cells only and negative controls consisting of media only were included in the assay. Ten percent (v/v) DMSO was used as a cytotoxic control in all experiments.

The cell viability data demonstrates that both blank and drug-loaded antihyphal liposomes maintain high vaginal epithelial cell viability across the concentration range tested. Cell viability following exposure to free drug at a highest concentration of 0.1 mg/mL was also evaluated, with a vehicle control of 1% (v/v) methanol included for free drug studies. The liposome formulations including 85:15 HSPC:CHOL, 83:15:2 HSPC:CHOL:FAR, and 80:15:5 HSPC:CHOL:FAR, both blank and drug-loaded, exhibited cell viability comparable to untreated controls.

The cytocompatibility data indicated that the incorporation of farnesol into the lipid bilayer does not induce cytotoxicity in vaginal epithelial cells. The drug-loaded liposome formulations containing fluconazole or miconazole also maintained cell viability, indicating that the encapsulated antifungal therapeutics do not cause cytotoxic effects when delivered via the liposome formulation. The liposome composition thus provided a biocompatible delivery system for treatment of vulvovaginal candidiasis without causing damage to vaginal epithelial tissue.

The method for FIG. 10 of the present disclosure provides that the cytotoxicity of liposomes was evaluated against vaginal epithelial cells using a CCK-8 assay. Vaginal epithelial cells were cultured in keratinocyte SFM supplemented with 0.1 ng mL⁻¹ rEGF, 0.05 mg mL⁻¹ BPE, and calcium chloride (44.1 mg L⁻¹) at 37° C. and 5% CO₂ until 80% confluent. Cells were seeded in tissue culture-treated 96-well plates at a density of 10,000 cells/cm₂ (~3,200 cells/well) and incubated for 24 h at 37° C. and 5% CO₂. Cells were then treated with serial dilutions of each liposome formulation (highest concentration=250 μg/mL) for ~16 h under the same conditions. The media was replaced with fresh media containing 10% (v/v) CCK-8 reagent, and plates were incubated for an additional 2 h at 37° C. and 5% CO₂ before measuring absorbance at 450 nm. Positive and negative controls (cells only and media only, respectively) were included. Cell viability following exposure to free drug (highest concentration=0.1 mg/mL) was also evaluated, with a vehicle control of 1% (v/v) MeOH included for free drug studies. Ten percent (v/v) DMSO was used as a cytotoxic control in all experiments.

### Example III

### Exploring the Efficacy of Nanoparticles in Enhancing Fungal Clearance In Vitro and In Vivo

In one embodiment, the present disclosure provides experiments for evaluating candidalysin neutralization upon exposure to anionic polymers.

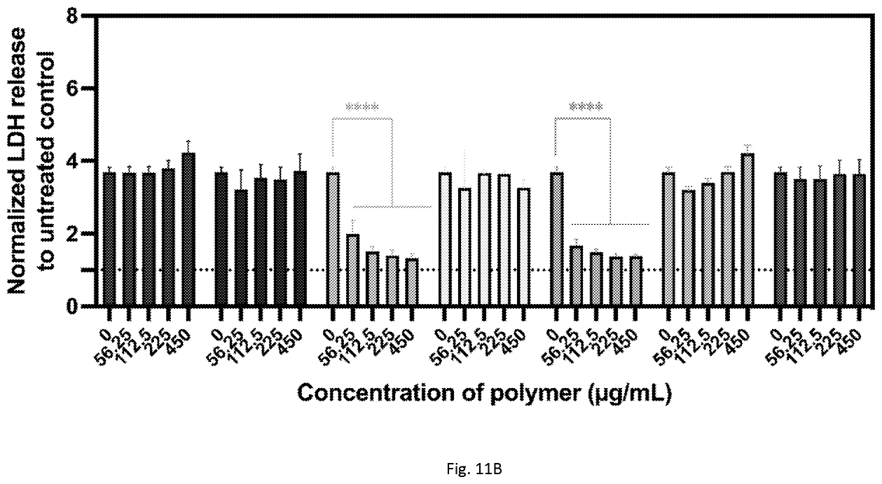

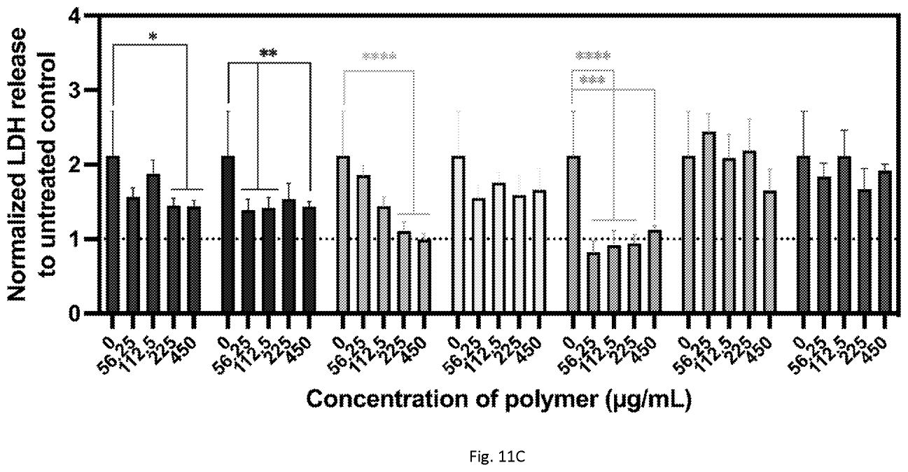

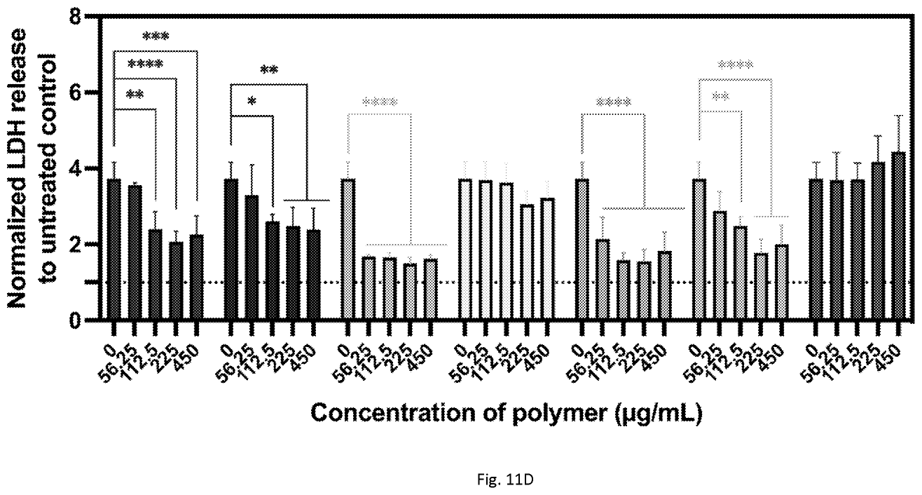

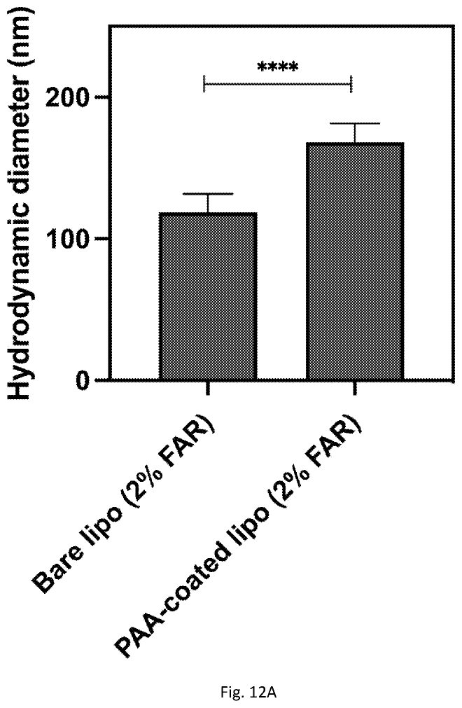

The method for FIG. 11 of the present disclosure provides that candidalysin neutralization upon exposure to anionic polymers was evaluated using a lactate dehydrogenase (LDH) assay in vaginal epithelial cells, fibroblasts, and human umbilical vein endothelial cells (HUVECs). Cells were seeded and plated at a density of 10,000 cells/cm² (~3,200 cells/well). Cells were exposed to 250 μg/mL of candidalysin and polymers at various concentrations (450, 225, 112.5, and 56.25 μg/mL). Neutralization of candidalysin was evaluated upon simultaneous addition of candidalysin and polymer. Following addition of the polymer-candidalysin mixture, cells were incubated overnight (~18 h) at 37° C. and 5% CO₂. Cytotoxicity was assessed using a LDH release assay, in which lactate dehydrogenase released from damaged or lysed cells into the culture supernatant was quantified spectrophotometrically according to the manufacturer's instructions. Positive controls of untreated cells and negative controls of media only and 1×PBS only were included for all methods.

As shown in FIG. 11A, the experimental design involved exposing cells to 250 μg/mL of candidalysin and polymers at various concentrations including 450, 225, 112.5, and 56.25 μg/mL. Neutralization of candidalysin was evaluated upon simultaneous addition of candidalysin and polymer to the cell cultures. Following addition of the polymer-candidalysin mixture, cells were incubated overnight for approximately 18 hours at 37° C. and 5% CO₂. Cytotoxicity was assessed using the LDH release assay, in which lactate dehydrogenase released from damaged or lysed cells into the culture supernatant was quantified spectrophotometrically according to the manufacturer's instructions.

As further shown in FIG. 11A, the well 1 configuration included positive controls of untreated cells and negative controls of media only and 1×PBS only. The experimental design allowed comparison of candidalysin-induced cytotoxicity in the presence and absence of neutralizing polymers. The polymers evaluated include poly(acrylic acid) (PAA) and heparin, which are negatively charged polymers capable of electrostatically interacting with the positively charged candidalysin peptide at physiological pH.

As shown in FIG. 11B, cytotoxicity to fibroblasts upon simultaneous exposure to candidalysin at 250 μg/mL and polymer of interest was measured through release of intracellular lactate dehydrogenase (LDH). The dotted line in FIG. 11B represents normal LDH release of untreated controls. The fibroblast cytotoxicity data demonstrated that PAA and heparin reduce candidalysin-induced LDH release in a concentration-dependent manner. Statistical analysis was performed using a two-way ANOVA with Tukey's post hoc analysis with n=4 and α=0.05.

As further shown in FIG. 11B, PAA at concentrations of 450 μg/mL and 225 μg/mL reduced candidalysin-induced cytotoxicity in fibroblasts to levels approaching the untreated control baseline. Heparin exhibited similar candidalysin-neutralizing activity in fibroblasts at equivalent concentrations. The negatively charged polymer coating may alternatively be heparin instead of PAA, though heparin has anticoagulant properties that may limit therapeutic use. The PAA coating was selected for the candidalysin-neutralizing liposomes based on the comparable neutralization activity and the absence of anticoagulant effects.

As shown in FIG. 11C, cytotoxicity to human umbilical vein endothelial cells (HUVECs) upon simultaneous exposure to candidalysin at 250 μg/mL and polymer of interest was measured through LDH release. The HUVEC cytotoxicity data demonstrates that PAA and heparin neutralize candidalysin-induced cytotoxicity in endothelial cells. The dotted line represents normal LDH release of untreated HUVEC controls. PAA at concentrations of 450 μg/mL and 225 μg/mL reduced candidalysin-induced LDH release in HUVECs to levels comparable to untreated controls.

As further shown in FIG. 11C, the candidalysin neutralization activity of PAA in HUVECs was comparable to the activity observed in fibroblasts. The concentration-dependent reduction in LDH release indicated that higher polymer concentrations provide greater protection against candidalysin-induced cell damage. The PAA coating neutralized and degraded candidalysin peptide secreted by hyphae through electrostatic interaction with the positively charged toxin. Candidalysin is positively charged at physiological pH, and the negatively charged carboxyl groups of PAA interact electrostatically with the cationic residues of the candidalysin peptide.

As shown in FIG. 11D, cytotoxicity to vaginal epithelial cells upon simultaneous exposure to candidalysin at 250 μg/mL and polymer of interest was measured through LDH release. The vaginal epithelial cell cytotoxicity data is relevant to the therapeutic application of the liposome composition for treatment of vulvovaginal candidiasis. The dotted line represents normal LDH release of untreated vaginal epithelial cell controls. PAA at concentrations of 450 μg/mL and 225 μg/mL reduced candidalysin-induced LDH release in vaginal epithelial cells.

As further shown in FIG. 11D, the candidalysin neutralization activity of PAA in vaginal epithelial cells supported the use of PAA-coated liposomes for treatment of vulvovaginal candidiasis. The electrostatic interaction between the negatively charged PAA coating and the positively charged candidalysin peptide resulted in neutralization of the pore-forming toxin activity. The PAA coating sequestered candidalysin molecules released by hyphal cells, preventing the toxin from inserting into epithelial cell membranes and forming cytolytic pores.

The candidalysin neutralization data from FIGS. 11B-11D demonstrated that PAA provides protection against candidalysin-induced cytotoxicity across multiple cell types including fibroblasts, HUVECs, and vaginal epithelial cells. The PAA-coated liposomes thus provided a dual-action therapeutic approach by combining the antihyphal activity of the farnesol-containing liposomes with the candidalysin-neutralizing activity of the PAA coating. The electrostatic interaction between PAA and candidalysin resulted in degradation of the candidalysin peptide structure, rendering the toxin inactive and preventing epithelial damage at the site of C. albicans infection.

In another embodiment, the present disclosure provides characterization of the effect of polymer coating on liposome size by measuring the hydrodynamic diameter of uncoated and PAA-coated liposomes. Each liposome of the plurality of liposomes further comprised a cationic lipid incorporated into the lipid bilayer. In some embodiments, the cationic lipid was 1,2-dioleoyl-3-trimethylammonium-propane (DOTAP). The cationic lipid DOTAP was incorporated at 5% (w/w) into the liposome formulation to generate a net positive surface charge for electrostatic polymer coating.

The liposome formulations and characterizations are summarized in Table 7.

TABLE 7
Summary of liposome formulations and physicochemical characterization.
Hydrodynamic diameter, ζ-potential, and PDI. Data presented as
mean ± SD (n = 3 biological replicates).

| | Hydrodynamic | | ζ-Potential |
|---|---|---|---|
| Formulation | Diameter (nm) | PDI | (mV) |
| 80:15:5 HSPC:CHOL: DOTAP | 122.48 ± 2.60 | 0.07 ± 0.01 | 28.83 ± 3.45 |
| 78:15:5:2 | 118.63 ± 14.43 | 0.08 ± 0.01 | 29.80 ± 4.78 |
| HSPC:CHOL:DOTAP:FAR | | | |

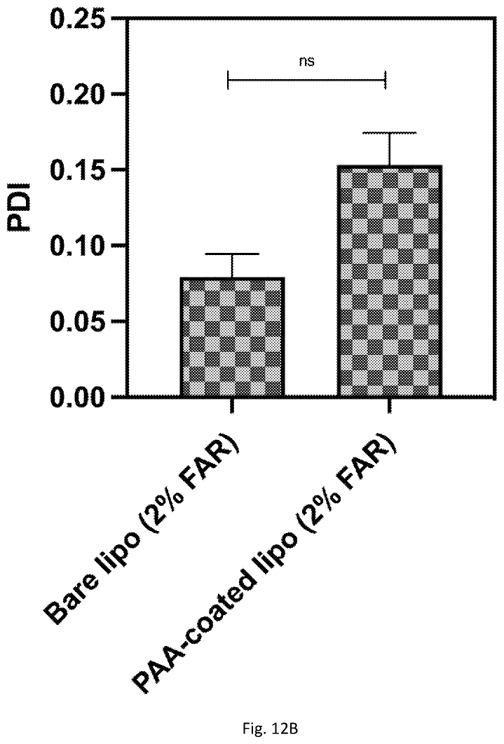

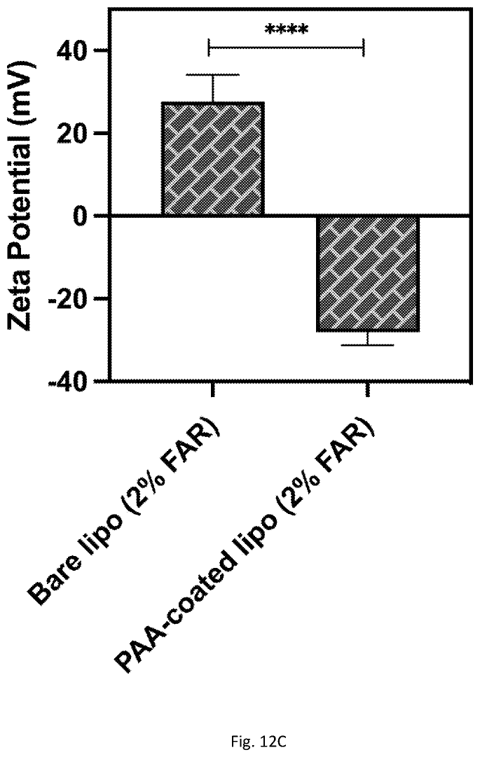

The methods for FIG. 12 of the present disclosure provides that PAA-coated liposomes were prepared by adding DOTAP-containing liposomes to the polymer solution in a 1:4 (v/v) ratio under magnetic stirring, resulting in a total volume of 5 mL. Liposomes were introduced dropwise using a peristaltic at a flow rate of 1.6 mL/min, followed by an additional 5 min of stirring after complete addition. The final liposome and polymer concentrations were 0.6 mM and 0.08% (w/v), respectively.

As shown in FIG. 12A, the 80:15:5 HSPC:CHOL:DOTAP formulation exhibited a hydrodynamic diameter of approximately 122.48±2.60 nm. The 78:15:5:2 HSPC:CHOL:DOTAP:FAR formulation exhibited a hydrodynamic diameter of approximately 118.63±14.43 nm. The liposome size is selected to be 100 nm or less to minimize unwanted immunologic responses, membrane disruption, and cellular toxicity. The hydrodynamic diameters of the DOTAP-containing formulations fall within the target size range of approximately 100 nm to 150 nm suitable for therapeutic applications.

As further shown in FIG. 12A, each liposome of the plurality of liposomes were further coated on an outer surface of the lipid bilayer with a negatively charged polymer. In some embodiments, the negatively charged polymer was poly(acrylic acid) (PAA). PAA was added dropwise to the liposome suspensions at a flow rate of 0.1 mL/min under constant stirring at 300 rpm with a micro stir bar. After PAA addition, the formulation undergoes sterile filtration and equilibration at 4° C. for 30 minutes to allow formation of a stable polymer-coated liposome.

As shown in FIG. 12B, the zeta potential of uncoated and PAA-coated liposomes was measured to characterize the surface charge before and after polymer coating. The 80:15:5 HSPC:CHOL:DOTAP formulation exhibited a zeta potential of approximately 28.83±3.45 mV, indicating a net positive surface charge due to the incorporation of the cationic lipid DOTAP. The 78:15:5:2 HSPC:CHOL:DOTAP:FAR formulation exhibited a zeta potential of approximately 29.80±4.78 mV. The positive surface charge enables electrostatic interaction with the negatively charged PAA polymer for coating.

As shown in FIG. 12B, PAA-coated liposomes were prepared by adding DOTAP-containing liposomes to the polymer solution in a 1:4 (v/v) ratio under magnetic stirring using a peristaltic pump at a flow rate of 1.6 mL/min. Liposomes are introduced dropwise using the peristaltic pump, followed by an additional 5 minutes of stirring after complete addition. The final liposome and polymer concentrations after PAA coating are 0.6 mM and 0.08% (w/v), respectively. The PAA coating resulted in a shift of the zeta potential from positive to negative values, confirming successful electrostatic adsorption of the negatively charged polymer onto the liposome surface.

As shown in FIG. 12C, the polydispersity index (PDI) of uncoated and PAA-coated liposomes was measured to assess the uniformity of size distribution following polymer coating. The 80:15:5 HSPC:CHOL:DOTAP formulation exhibited a PDI of approximately 0.07±0.01. The 78:15:5:2 HSPC:CHOL:DOTAP:FAR formulation exhibited a PDI of approximately 0.08±0.01. The low PDI values indicated that the DOTAP-containing liposome formulations maintain uniform size distribution comparable to the non-cationic formulations described previously.

As further shown in FIG. 12C, the PAA coating process did not substantially increase the polydispersity of the liposome formulations. The electrostatic adsorption of PAA onto the positively charged liposome surface resulted in stable polymer-coated liposomes with maintained size uniformity. Statistical analysis was performed using a two-way ANOVA followed by Tukey's multiple comparisons test with α=0.05. The characterization data from FIGS. 12A-12C demonstrated that the PAA-coated liposomes exhibit suitable physicochemical properties for the dual-action therapeutic approach combining antihyphal activity with candidalysin neutralization.

In another embodiment, the present disclosure provides evaluation of the PAA coating stability under varying pH conditions to assess the robustness of the polymer-coated liposome formulation across a range of physiological and environmental pH values. The PAA-coated liposomes remained stable across physiologically relevant pH conditions, particularly within the vaginal pH range of approximately 3.8-5. While increases in hydrodynamic diameter and PDI were observed at the more extreme conditions tested (pH 3 and pH 8), the formulations maintained stability within the pH range most relevant to the intended therapeutic environment. These results suggested that the liposomes were sufficiently stable for applications in the vaginal environment despite reduced stability at non-physiological pH extremes.

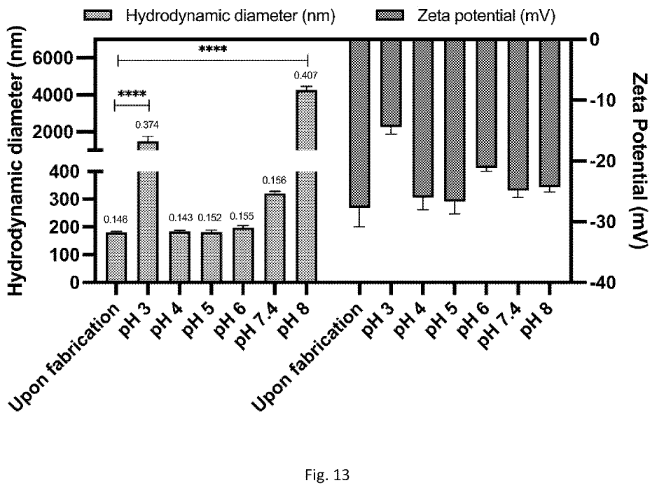

The method for FIG. 13 of the present disclosure provides that the pH stability of PAA-coated liposomes was evaluated by incubating 600 μL of liposomes (2% FAR) with 1 mL of 10 mM HEPES buffer adjusted to pH 3, 4, 5, 6, 7.4, or 8. The mixtures were homogenized by pipetting up and down and inverting the tubes several times. Samples were incubated at 37° C. with shaking at 100 rpm for approximately 2 hours. Following incubation, DLS measurements were performed to assess the hydrodynamic diameter, polydispersity, and overall colloidal stability of the liposomes under each pH condition.

As shown in FIG. 13, the pH stability of PAA-coated liposomes was evaluated by incubating 600 μL of liposomes containing 2% FAR with 1 mL of 10 mM HEPES buffer adjusted to pH 3, 4, 5, 6, 7.4, or 8. The mixtures were homogenized by pipetting up and down and inverting the tubes several times to ensure uniform distribution. Samples were incubated at 37° C. with shaking at 100 rpm for approximately 2 hours to simulate physiological temperature conditions and provide agitation representative of in vivo environments.

As further shown in FIG. 13, DLS measurements were performed following incubation to assess the hydrodynamic diameter, polydispersity, and overall colloidal stability of the liposomes under each pH condition. The hydrodynamic diameter measurements show that the PAA-coated liposomes remain relatively consistent in size under moderately acidic conditions, particularly within the physiologically relevant vaginal pH range. While increases in hydrodynamic diameter were observed at the extreme pH conditions tested (pH 3 and pH 8), the liposomes maintained stable size at pH values between approximately 4 and 5. These results indicated that the PAA coating remains associated with the liposome surface under the pH conditions most relevant to the intended therapeutic environment.

The polydispersity index for each formulation was indicated in FIG. 13 above the corresponding bar for each pH condition. The PDI values indicated that the PAA-coated liposomes maintain a relatively uniform size distribution within the physiologically relevant vaginal pH range. Although increases in PDI were observed at pH 3 and pH 8, suggesting reduced colloidal stability at these extremes, the liposomes remained well-dispersed at pH values between approximately 4 and 5. This stability was particularly relevant for intravaginal administration, as the vaginal environment typically exhibits pH values ranging from approximately 3.8 to 5 under normal conditions.

With continued reference to FIG. 13, the PAA-coated liposomes exhibited reduced stability at neutral and basic conditions (pH 7.4-8), as indicated by increases in hydrodynamic diameter and PDI. Although these changes suggested some loss of colloidal stability at higher pH values, this did not significantly impact the intended application, as the formulations remained stable within the physiologically relevant vaginal pH range. Because the target therapeutic environment typically exhibits pH values between approximately 3.8 and 5, the observed instability at pH 7.4-8 was not expected to limit intravaginal administration. These results suggested that while electrostatic interactions between the positively charged DOTAP-containing liposome surface and the negatively charged PAA polymer coating may weaken at pH extremes, they remained sufficiently robust within the acidic pH conditions relevant to the intended therapeutic environment.

The pH stability data indicated that partial destabilization occurs at the most extreme pH conditions tested (pH 3 and pH 8), likely due to pH-dependent changes in polymer ionization and electrostatic interactions. The carboxyl groups of PAA have a pKa of approximately 4.5, and the polymer remains partially ionized even at pH 3, allowing maintenance of electrostatic interactions with the cationic liposome surface. Importantly, the PAA-coated liposomes maintained colloidal stability within the vaginally relevant pH range (~3.8-5), supporting the therapeutic utility of the dual-action liposome composition for treatment of fungal infections in this environment despite reduced stability at non-physiological pH extremes.

According to some embodiments, the present disclosure provides encapsulation of three antifungal drugs including Posaconazole, Fluconazole, Voriconazole, as examples. More therapeutic drugs can be encapsulated. The structure and characteristics of the exemplary drugs are indicated below.

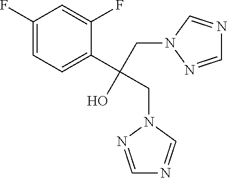 [SMILES: OC(Cn1cncn1)(Cn1cncn1)c1ccc(F)cc1F]

Fluconazolel
MW: 306.3 g/mol
log P: 0.5

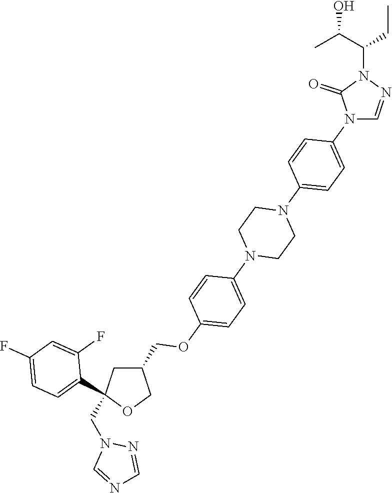 [SMILES: CC[C@@H]([C@H](C)O)n1ncn(-c2ccc(N3CCN(c4ccc(OC[C@@H]5CO[C@@](Cn6cncn6)(c6ccc(F)cc6F)C5)cc4)CC3)cc2)c1=O]

Posoconazolel
MW: 700.7 g/mol
log P: 5.5

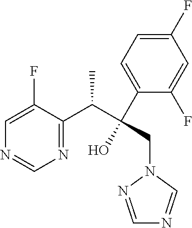 [SMILES: C[C@@H](c1ncncc1F)[C@](O)(Cn1cncn1)c1ccc(F)cc1F]

Voriconazole1
MW: 349.3 g/mol
log P: 1.82

According to other embodiments, the present disclosure provides liposomes encapsulating antifungal drugs commonly used in treatment of vulvovaginal candidiasis, including the following two antifungal drugs.

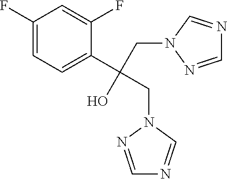 [SMILES: OC(Cn1cncn1)(Cn1cncn1)c1ccc(F)cc1F]

Fluconazolel
MW: 306.3 g/mol
log P: 0.5, and

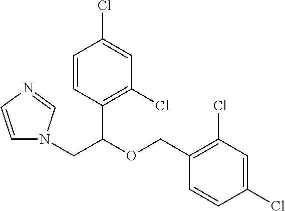 [SMILES: Clc1ccc(COC(Cn2ccnc2)c2ccc(Cl)cc2Cl)c(Cl)c1]

Miconazolel
MW: 416.1 g/mol
log P: 5.3

### Equivalents

Other embodiments will be evident to those of skill in the art. It should be understood that the foregoing description is provided for clarity only and is merely exemplary. The spirit and scope of the present invention are not limited to the above example but are encompassed by the claims. All publications, patents, and patent applications cited above are incorporated by reference herein in their entirety for all purposes to the same extent as if each individual publication or patent application were specifically indicated to be so incorporated by reference.

A number of implementations have been described. Nevertheless, it will be understood that various modifications may be made without departing from the spirit and scope of the disclosure. Accordingly, other implementations are within the scope of the following claims.

1. A liposome composition comprising a plurality of liposomes wherein each liposome of the plurality of liposomes comprises a lipid bilayer comprising hydrogenated soy phosphatidylcholine (HSPC), cholesterol (CHOL), and/or farnesol (FAR).

2. The liposome composition of claim 1, wherein the molar ratios of HSPC:CHOL:FAR comprise HSPC: 78-85 mol %, CHOL: 15-40 mol %, and FAR: 0-5 mol %.

3. The liposome composition of claim 1, wherein each liposome of the plurality of liposomes is further coated on the outer lipid bilayer with a poly(acrylic acid) (PAA), heparin, and/or other polyanions.

4. The liposome composition of claim 1, wherein each liposome of the plurality of liposomes has a size of 50 nm-200 nm in diameter.

5. (canceled)

6. The liposome composition of claim 1, wherein each liposome of the plurality of liposomes is further encapsulated with one or more antifungal therapeutics comprising azole, polyene, or enchinocandin.

7. The liposome composition of claim 6, wherein the azole comprises posaconazole, fluconazole, miconazole, and voriconazole.

8. The liposome composition of claim 6, wherein the antifungal therapeutics are encapsulated within the lipid bilayer and/or the hydrophilic core of each liposome of the plurality of liposomes.

9. The liposome composition of claim 1, wherein each liposome of the plurality of liposomes is further labeled with a fluorescent dye.

10. A pharmaceutical composition comprising a liposome composition of claim 1 and a pharmaceutically acceptable excipient.

11. (canceled)

12. A kit comprising the liposome composition of claim 1, further comprising technical instructions providing information on administration and dosage of the composition.

13. A method of treating or preventing a fungal infection within a subject in need thereof, comprising administering to the subject a therapeutically effective amount of the liposome composition of claim 1.

14. (canceled)

15. (canceled)

16. (canceled)

17. (canceled)

18. (canceled)

19. (canceled)

20. (canceled)

21. (canceled)

22. The method of claim 13, wherein the liposome composition is administered to the subject by a route selected from the group consisting of mucosal, intranasal, intravaginal, intramuscular, intravenous, subcutaneous, intradermal, subdermal, and transdermal routes of administration.

23. The method of claim 13, wherein the subject is a human.

24. The method of claim 13, wherein the fungal infection comprises a yeast infection, invasive candidiasis, or vulvovaginal candidiasis caused by an overgrowth of C. albicans.

25. (canceled)

26. (canceled)

27. The method of claim 13, wherein the liposome composition inhibits hyphal formation or yeast-to-hyphal transition in C. albicans.

28. (canceled)

29. The method of claim 15, wherein the outer polymer coating interacts with and neutralizes candidalysin peptide secreted by existing hyphae.

30. (canceled)

31. (canceled)

32. (canceled)

33. The method of claim 13, wherein, after administration, the liposome composition contacts fungal biofilm in the subject and disrupts the fungal biofilm.

34. (canceled)

35. A method of making a liposome formulation, comprising
1) providing a lipid film over a drug film, wherein the lipid film comprises a desired molar ratio of HSPC, CHOL, and/or FAR, and wherein the drug film comprises one or more antifungal therapeutics,
2) hydrating the lipid films with 10 mM HEPES buffer (pH 7.4) to form a lipid suspension with a total lipid concentration of 2.5 mg/mL, followed by vigorous vortexing to generate multilamellar vesicles,
3) sonicating the lipid suspension for 15 min at 65° C., and
4) extruding through a 100 nm polycarbonate membrane to obtain uniformly sized liposomes.

36. (canceled)

37. The method of claim 35, wherein the liposome formulation is further labeled with a fluorescent dye.

38. (canceled)

39. (canceled)

40. (canceled)

41. (canceled)

42. A method of treating a fungal biofilm within a subject in need thereof, comprising administrating to the subject a pharmaceutical composition of claim 10, wherein the farnesol, antifungal therapeutics, and PAA in the composition prevent hyphal formation, interact with candidalysin peptide secreted by the fungal hyphae, and prevent or disrupt the formation of the fungal biofilm.
